(19) United States
(12) Patent Application Publication
(10) Pub. No.: US 2019/0169657 A1
(43) Pub. Date: Jun. 6, 2019

(54) METHYLENEMALONIC ACID AND INTERMEDIATES, PROCESSES FOR THEIR PREPARATION AND ENGINEERED MICROORGANISMS

(71) Applicant: LCY BIOSCIENCES INC., SARNIA (CA)

(72) Inventors: MAN KIT LAU, MINNEAPOLIS, MN (US); CATHY STALOCH HASS, SAINT LOUIS PARK, MN (US); JAMES R. MILLIS, PLYMOUTH, MN (US)

(21) Appl. No.: 16/321,181

(22) PCT Filed: Jul. 28, 2017
(86) PCT No.: PCT/CA2017/050903

§ 371 Date: Jan. 28, 2019

Related U.S. Application Data
(60) Provisional application No. 62/367,833, filed on Jul. 28, 2016.

(51) Int. Cl. C12P 7/46; C12P 7/62; C07C 59/325; C12R 1/645

(52) U.S. Cl. CPC C12P 7/46; C12P 7/62; C12Y 301/02001; C12R 1/645; C12Y 604/01002; C07C 59/325

(57) ABSTRACT

The description relates to, inter alia, recombinant microorganisms, engineered metabolic pathways, chemical catalysts, and products produced through the use of the described methods and materials. The products produced include methylenemalonic acid and intermediates, as well as their salts and esters.

### RELATED APPLICATION

This application claims priority under the applicable law to U.S. provisional application No. 62/367,833 filed on Jul. 28, 2016, the content of which is incorporated herein by reference in its entirety for all purposes.

### TECHNICAL FIELD

The description provides, inter alia, recombinant microorganisms, engineered metabolic pathways, chemical catalysts, and products produced through the use of the described methods and materials. The products produced include methylenemalonic acid, intermediates as well as their salts and esters.

### BACKGROUND

Currently, many carbon-containing chemicals are derived from petroleum based sources. Reliance on petroleum-derived feedstocks contributes to depletion of petroleum reserves and the harmful environmental impact associated with oil drilling.

Certain carbonaceous products of sugar fermentation are seen as replacements for petroleum-derived materials for use as feedstocks for the manufacture of carbon-containing chemicals. Such products include intermediates used in the production of chemical building blocks such as methylenemalonic acid. Precursors of methylenemalonic acid production are petroleum-derived. Research regarding a bio-based route is currently ongoing but is not yet commercialized. Methylenemalonic acid and their salts and esters represent a growing market for which all commercial production today is petroleum-derived.

### SUMMARY

The present application generally relates to methylenemalonic acid, i.e. the compound of Formula I:

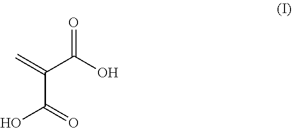

or a salt or ester thereof.

The present application also further relates to methods for the preparation of the compound of Formula I, or a salt or ester thereof, via biosynthetic or semi-synthetic pathways and to recombinant microorganisms for use in such methods.

According to one aspect, the present application relates to a recombinant microorganism comprising 2-hydroxymethylmalonic acid, and at least one recombinant nucleic acid sequence encoding at least one enzyme selected from a CoA carboxylase and a CoA hydrolase, wherein the 2-hydroxymethylmalonic acid is a compound of Formula II:

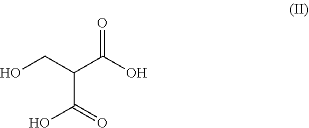

or a salt or ester thereof.

In one embodiment, the recombinant microorganism further comprises 3-hydroxypropionyl-CoA.

In another embodiment, the recombinant microorganism selectively overproduces 2-hydroxymethylmalonic acid, or a salt or ester thereof. For instance, the recombinant microorganism produces at least 0.1 g/L/hour of 2-hydroxymethylmalonic acid or a salt or ester thereof, e.g. at least 0.1 g/L/hour of 2-hydroxymethylmalonic acid. In another embodiment, the recombinant microorganism further comprises a recombinant nucleic acid sequence encoding an organic acid transporter. In another embodiment, the application relates to a method for making 2-hydroxymethylmalonic acid or a salt or ester thereof, comprising culturing the recombinant microorganism in the presence of a carbon source (e.g. a carbohydrate); and isolating the 2-hydroxymethylmalonic acid or its salt or ester.

According to another aspect, the present application relates to a recombinant microorganism comprising 2,3-dioxobutyric acid or acetoacetic acid or a salt or ester thereof and at least one recombinant nucleic acid sequence encoding at least one enzyme selected from a CoA-hydrolase, a thiolase and an alcohol dehydrogenase. In one embodiment, the recombinant microorganism selectively overproduces 2,3-dioxobutyric acid or acetoacetic acid, or a salt or ester thereof. For example, the recombinant microorganism produces at least 0.1 g/L/hour of 2,3-dioxobutyric acid or acetoacetic acid, or a salt or ester thereof, e.g. at least 0.1 g/L/hour of 2,3-dioxobutyric acid or acetoacetic acid. In one embodiment, the recombinant microorganism further comprises a recombinant nucleic acid sequence encoding an organic acid transporter. According to an embodiment, the application further relates to a method for making 2,3-dioxobutyric acid or acetoacetic acid or a salt or ester thereof, comprising culturing the recombinant microorganism in the presence of a carbon source (e.g. a carbohydrate); and isolating the 2,3-dioxobutyric acid or acetoacetic acid or a salt or ester thereof.

According to another aspect, the application relates to a recombinant microorganism comprising methylmalonic acid and at least one recombinant nucleic acid sequence encoding at least one enzyme selected from a CoA carboxylase and a CoA hydrolase, wherein said methylmalonic acid is a compound of Formula IV:

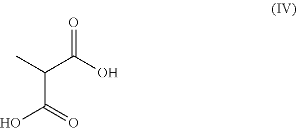

or a salt or ester thereof.

In one embodiment, the recombinant microorganism further comprises propionyl-CoA or a salt or ester thereof. In another embodiment, the recombinant microorganism further comprises 2-oxobutyrate or a salt or ester thereof. According to another embodiment, the recombinant microorganism selectively overproduces methylmalonic acid, or a salt or ester thereof. For instance, the recombinant microorganism produces at least 0.1 g/L/hour of methylmalonic acid, or a salt or ester thereof, e.g. at least 0.1 g/L/hour of methylmalonic acid. In a further embodiment, the recombinant microorganism further comprises a recombinant nucleic acid sequence encoding an organic acid transporter. According to a further embodiment, provided is a method for making methylmalonic acid, or a salt or ester thereof, comprising culturing the recombinant microorganism in the presence of a carbon source (e.g. a carbohydrate or amino acid); and separating the methylmalonic acid, or it salt or ester. For instance, the carbon source comprises an amino acid selected from threonine, homoserine and methionine.

According to a further aspect, the present application relates to a recombinant microorganism comprising methylenemalonic acid or a salt or ester thereof, and at least one recombinant nucleic acid sequence encoding at least one enzyme selected from a transaminase, a synthase, an alcohol dehydrogenase, a semialdehyde dehydrogenase, a dehydratase and a decarboxylase. In one embodiment, the recombinant microorganism further comprises 1,1,2-ethenetricarboxylic acid and/or 1-hydroxy-1,1,2-ethanetricarboxylic acid and/or 1-formyl-1-hydroxy-1,2-ethanedicarboxylic acid and/or itatartaric acid. In another embodiment, the recombinant microorganism selectively overproduces methylenemalonic acid, or a salt or ester thereof. For instance, the recombinant microorganism produces at least 0.1 g/L/hour of methylenemalonic acid, or a salt or ester thereof, e.g. at least 0.1 g/L/hour of methylenemalonic acid. In yet another embodiment, the recombinant microorganism further comprises a recombinant nucleic acid sequence encoding an organic acid transporter. According to a further embodiment, provided is a method for making methylenemalonic acid or a salt or ester thereof, comprising culturing the recombinant microorganism in the presence of a carbon source (e.g. a carbohydrate); and isolating the methylenemalonic acid or its salt ester.

In one embodiment, the recombinant microorganism herein defined is a prokaryote. For instance, the microorganism is selected from Escherichia coli (E. coli), Enterobacter, Azotobacter, Erwinia, Bacillus, Pseudomonas, Klebsiella, Proteus, Salmonella, Serratia, Shigella, Rhizobia, Vitreoscilla, and Paracoccus. In another embodiment, the recombinant microorganism herein defined is a eukaryote (e.g., a yeast or a fungus). For example, the microorganism is selected from Candida, Pichia, Saccharomyces, Schizosaccharomyces, Zygosaccharomyces, Kluyveromyces, Debaryomyces, Pichia, Issatchenkia, Yarrowia, Hansenula, Aspergillus, and Ustilago. For instance, the microorganism is a host yeast cell selected from C. sonorensis, K. marxianus, K. thermotolerans, C. methanesorbosa, Saccharomyces bulderi (S. bulden), I. orientalis, C. lambica, C. sorboxylosa, C. zemplinina, C. geochares, P. membranifaciens, Z. kombuchaensis, C. sorbosivorans, C. vanderwaltii, C. sorbophila, Z. bisporus, Z. lentus, Saccharomyces bayanus (S. bayanus), D. castellii, C, C. etchellsii, K. lactis, P. jadinii, P. anomala, Saccharomyces cerevisiae (S. cerevisiae), Pichia galeiformis, Pichia sp. YB-4149 (NRRL designation), Candida ethanolica, P. deserticola, P. membranifaciens, P. fermentans and Saccharomycopsis crataegensis (S. crataegensis). In addition, the fungi may include Aspergillus niger, Aspergillus terreus, Aspergillus oryzae, Ustilago maydis, Ustilago cynodontis, or other fungi.

According to another aspect, the present application relates to a method for making a methylenemalonic acid of Formula I:

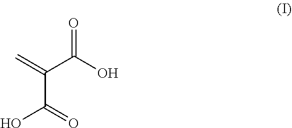

or a salt or ester thereof;

the method comprising treating a compound of Formula II:

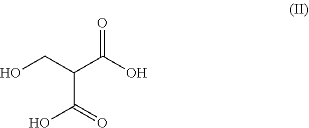

or a salt or ester thereof;

by heating and/or contacting with a catalyst to dehydrate the compound of Formula II to produce a compound of Formula I, or its salt or ester. In one embodiment, the method further comprises making a compound of Formula II, comprising the steps of culturing a recombinant microorganism as herein defined in the presence of a carbon source (e.g. a carbohydrate); and isolating the compound of Formula II.

According to a further aspect, the application relates to a method for making a methylenemalonic acid of Formula I:

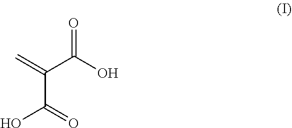

or a salt or ester thereof;

the method comprising treating a methyltartronic acid of Formula III:

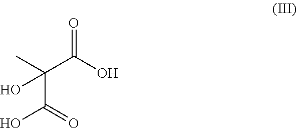

or a salt or ester thereof;

by heating and/or contacting with a catalyst, optionally followed by pyrolysis, to dehydrate the compound of Formula III and/or contacting with a bromination agent followed by an elimination agent such as a base, to produce methylenemalonic acid or a salt or ester thereof. In one embodiment, the method further comprises preparing methyltartronic acid or a salt or ester thereof, the preparation comprising the steps of chemically modifying a 2,3-dioxobutyric acid or acetoacetic acid produced by culturing a recombinant microorganism as herein defined in the presence of a carbon source (e.g. a carbohydrate); and isolating the compound of Formula III.

According to yet a further aspect, the application relates to a method for making a methylenemalonic acid of Formula I:

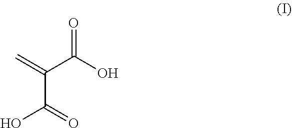

or a salt or ester thereof;

the method comprising treating a compound of Formula IV:

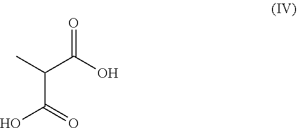

or a salt or ester thereof;

by heating in the presence of O₂ and/or contacting with a catalyst to dehydrogenate the compound of Formula IV to produce the methylenemalonic acid or a salt or ester thereof. In one embodiment, the method further comprises making a compound of Formula IV, comprising the steps of culturing a recombinant microorganism as herein defined in the presence of a carbon source (e.g. a carbohydrate); and separating the compound of Formula IV.

The present application also further relates to a compound of Formula V:

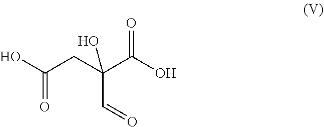 [SMILES: O=CC(O)(CC(=O)O)C(=O)O]

or a salt or ester thereof and to recombinant microorganisms and methods for their preparation.

### BRIEF DESCRIPTION OF THE FIGURES

FIG. 1 shows a general scheme showing various biosynthetic and semi-synthetic pathways to methylenemalonic acids according to embodiments of the present application.

FIG. 2 shows a semi-synthetic pathway useful for making methylenemalonic acids via chemical dehydration of 2-hydroxymethylmalonic acids, according to one embodiment.

FIG. 3 shows a semi-synthetic pathway useful for making methylenemalonic acids via chemical dehydration of methyltartronic acid, according to another embodiment.

FIG. 4 shows a semi-synthetic pathway useful for making methylenemalonic acids via chemical dehydrogenation of methylmalonic acids, according to another embodiment.

FIG. 5 shows a semi-synthetic pathway useful for making methylenemalonic acids via chemical dehydrogenation of methylmalonic acids starting from amino acids, according to another embodiment.

FIG. 6 shows a biosynthetic (fully biological) pathway useful for making methylenemalonic acids, according to a further embodiment.

FIG. 7 shows the relative growth of host organisms after 24 hours in the presence of alpha-hydroxymethyl-3-hydroxypropionic acid.

FIG. 8 shows CoA carboxylase activity of RpPCC lysate overtime.

FIG. 9 shows hydrolase activity of E coli lysate overexpressing TesB with 3HP-CoA and HMMCoA.

### DETAILED DESCRIPTION

### Definitions

General methods for molecular biology procedures and recipes for buffers, solutions, and media in the following examples are described in J. Sambrook, and D. W. Russell, Molecular Cloning: A Laboratory Manual, 3rd Ed., Cold Spring Harbor Laboratory Press, Cold Spring Harbor, N.Y., 2001. When listed, instructions from individual manufactures were used for some of the procedures. Restriction enzymes were purchased from New England Biolabs (Ipswich, Mass.), unless otherwise stated, and used in appropriate buffers as suggested by the manufacture. All chemicals were purchased from Sigma Aldrich (St. Louis, Mo.), unless otherwise specified.

For the purposes of this application, “native” as used herein with regard to a metabolic pathway refers to a metabolic pathway that exists and is active in the wild-type host strain. Genetic material such as coding regions, genes, promoters and terminators is “native” for purposes of this application if the genetic material has a sequence identical to (apart from individual-to-individual mutations which do not affect function) a genetic component that is present in the genome of the wild-type host cell (i.e., the exogenous genetic component is identical to an endogenous genetic component).

For the purposes of this description, genetic material such as a coding region, a gene, a promoter and a terminator is “endogenous” to a cell if it is (i) native to the cell, (ii) present at the same location as that genetic material is present in the wild-type cell and (iii) under the regulatory control of its native promoter and its native terminator and (iv) has not been altered directly or through a directed selection process.

For the purposes of this application, genetic material such as coding sequence, genes, promoters and terminators are “exogenous” to a cell if they are (i) non-native to the cell and/or (ii) are native to the cell, but are present at a location different than where that genetic material is present in the wild-type cell and/or (iii) are under the regulatory control of a non-native promoter and/or non-native terminator. Extra copies of native genetic material are considered as “exogenous” for purposes of this description, even if such extra copies are present at the same locus as that genetic material is present in the wild-type host strain and/or (iv) they are altered directly or through a selection process.

As used herein, the term “control sequences” included enhancer sequences, terminator sequences and promoters. As used herein “promoter” refers to an untranslated sequence located upstream (i.e., 5′) to the translation start codon of a gene (generally a sequence of about 1 to 1500 base pairs (bp), preferably about 100 to 1000 bp and especially of about 200 to 1000 bp) which controls the start of transcription of the gene. Where the promoters are non-native, they may be identical to or share a high degree of sequence identity (i.e., at least about 80%, at least about 85%, at least about 90%, at least about 95%, or at least about 99%) with one or more native promoters. Other suitable promoters and terminators include those described, for example, in WO99/14335, WO00/71738, WO02/42471, WO03/102201, WO03/102152 and WO03/049525.

The term “terminator” as used herein refers to an untranslated sequence located downstream (i.e., 3′) to the translation termination codon of a gene (generally a sequence of about 1 to 1500 bp, preferably of about 100 to 1000 bp, and especially of about 200 to 500 bp) which controls the end of transcription of the gene. Examples of terminators that may be linked to one or more exogenous genes in the yeast cells provided herein include, but are not limited to, terminators for PDC1, XR, XDH, transaldolase (TAL), transketolase (TKL), ribose 5-phosphate ketol-isomerase (RKI), CYB2, or iso-2-cytochrome c (CYC) genes or the galactose family of genes (especially the GAL 10 terminator), as well as any of those described in the various Examples that follow. Where the terminators are non-native, they may be identical to or share a high degree of sequence identity (i.e., at least about 80%, at least about 85%, at least about 90%, at least about 95%, or at least about 99%) with one or more native terminators.

A promoter or terminator is “operatively linked” to a coding sequence if its position in the genome relative to that of the coding sequence is such that the promoter or terminator, as the case may be, performs its transcriptional control function. One of ordinary skill in the art will also appreciate that the DNA sequence can include regions that give rise to RNA sequences that modulate translation.

“Increasing or decreasing” activity with regard to enzyme activities refers to the activity either being greater than that enzymatic activity found in the wild type strain (increasing activity), or refers to the activity being less than that enzymatic activity found in the wild type strain (decreasing activity or otherwise referred to as attenuating). One ordinarily skilled in the art will appreciate that the modulation of activity can be accomplished by (i) controlling polypeptide: polypeptide interactions, (ii) polypeptide: metabolite interactions (feedback inhibition), (iii) polypeptide/nucleic acid interactions, (iv) modifying the amino acid sequence to increase enzymatic activity and (iiv) nucleic acid interactions.

“Deletion or disruption” with regard to a gene means that either the entire coding region of the gene is eliminated (deletion) or the coding region of the gene, its promoter, and/or its terminator region is modified (such as by deletion, insertion, or mutation) such that the gene no longer produces an active enzyme, produces a severely reduced quantity of enzyme (at least 75% reduction, preferably at least 85% reduction, more preferably at least 95% reduction), or produces an enzyme with severely reduced (at least 75% reduced, preferably at least 85% reduced, more preferably at least 95% reduced) activity. A deletion or disruption of a gene can be accomplished by, for example, forced evolution, mutagenesis or genetic engineering methods, followed by appropriate selection or screening to identify the desired mutants.

“Overexpress” means the artificial expression of an enzyme in increased quantity. Overexpression of an enzyme may result from the presence of one or more exogenous gene(s), genetic engineering to increase the expression of the endogenous gene, or from other conditions. For purposes of this technologie, a yeast cell containing at least one exogenous gene is considered to overexpress the enzyme(s) encoded by such exogenous gene(s).

A “recombinant microorganism” is a microorganism, either eukaryotic or prokaryotic, that has a nucleotide sequence that has been altered by human intervention to include a sequence that is not the same as that found in the progenitor microorganism. One of ordinary skill the art will appreciate that such nucleic acid sequence alterations can be introduced through a variety of methods, including for example, mutation and selection, transformation, mating, homologous recombination and the like. Any method known in the art can be used to generate such recombinant microorganism. Moreover, the nucleic acid sequence alteration can be chromosomal or extrachromosomal.

A recombinant eukaryotic cell can be a yeast or a fungal cell comprising certain genetic modifications. The host yeast or fungi cell is one which as a wild-type strain is natively capable of metabolizing at least one sugar to pyruvate. Suitable host yeast cells include (but are not limited to) yeast cells classified under the genera Candida, Pichia, Saccharomyces, Schizosaccharomyces, Zygosaccharomyces, Kluyveromyces, Debaryomyces, Pichia, Issatchenkia, Yarrowia and Hansenula. Examples of host yeast cells include C. sonorensis, K. marxianus, K. thermotolerans, C. methanesorbosa, Saccharomyces bulderi (S. bulden), I. orientalis, C. lambica, C. sorboxylosa, C. zemplinina, C. geochares, P. membranifaciens, Z. kombuchaensis, C. sorbosivorans, C. vanderwaltii, C. sorbophila, Z. bisporus, Z. lentus, Saccharomyces bayanus (S. bayanus), D. castellii, C, boidinii, C. etchellsii, K. lactis, P. jadinii, P. anomala, Saccharomyces cerevisiae (S. cerevisiae), Pichia galeiformis, Pichia sp. YB-4149 (NRRL designation), Candida ethanolica, P. deserticola, P. membranifaciens, P. fermentans and Saccharomycopsis crataegensis (S. crataegensis). Suitable strains of K. marxianus and C. sonorensis include those described in WO 00/71738 A1, WO 02/42471 A2, WO 03/049525 A2, WO 03/102152 A2 and WO 03/102201A2. Suitable strains of I. orientalis are ATCC strain 32196 and ATCC strain PTA-6648. In addition, fungi may include Aspergillus niger, Aspergillus terreus, Aspergillus oryzae, Ustilago maydis, Ustilago cynodontis, or other fungi.

In some embodiments, the host cell is Crabtree negative as a wild-type strain. The Crabtree effect is defined as the occurrence of fermentative metabolism under aerobic conditions due to the inhibition of oxygen consumption by a microorganism when cultured at high specific growth rates (long-term effect) or in the presence of high concentrations of glucose (short-term effect). Crabtree negative phenotypes do not exhibit this effect, and are thus able to consume oxygen even in the presence of high concentrations of glucose or at high growth rates.

Modifications (insertion, deletions and/or disruptions) to the genome of the host cell described herein can be performed using methods known in the art. Exogenous genes may be integrated into the genome in a targeted or a random manner using, for example, well known electroporation and chemical methods (including calcium chloride and/or lithium acetate methods). In those embodiments where an exogenous gene is integrated in a targeted manner, it may be integrated into the locus for a particular native gene, such that integration of the exogenous gene is coupled with deletion or disruption of a native gene. Alternatively, the exogenous gene may be integrated into a portion of the native genome that does not correspond to a gene. Methods for transforming a yeast cell with an exogenous construct are described in, for example, WO99/14335, WO00/71738, WO02/42471, WO03/102201, WO03/102152, WO03/049525, WO2007/061590, WO 2009/065778 and PCT/US2011/022612. Insertion of exogenous genes is generally performed by transforming the cell with one or more integration constructs or fragments. The terms “construct” and “fragment” are used interchangeably herein to refer to a DNA sequence that is used to transform a cell. The construct or fragment may be, for example, a circular plasmid or vector, a portion of a circular plasmid or vector (such as a restriction enzyme digestion product), a linearized plasmid or vector, or a PCR product prepared using a plasmid or genomic DNA as a template. An integration construct can be assembled using two cloned target DNA sequences from an insertion site target. The two target DNA sequences may be contiguous or non-contiguous in the native host genome. In this context, “non-contiguous” means that the DNA sequences are not immediately adjacent to one another in the native genome, but instead are separated by a region that is to be deleted. “Contiguous” sequences as used herein are directly adjacent to one another in the native genome. Where targeted integration is to be coupled with deletion or disruption of a target gene, the integration construct also functions as a deletion construct. In such an integration/deletion construct, one of the target sequences may include a region 5′ to the promoter of the target gene, all or a portion of the promoter region, all or a portion of the target gene coding sequence, or some combination thereof. The other target sequence may include a region 3′ to the terminator of the target gene, all or a portion of the terminator region, and/or all or a portion of the target gene coding sequence. Where targeted integration is not to be coupled to deletion or disruption of a native gene, the target sequences are selected such that insertion of an intervening sequence will not disrupt native gene expression. An integration or deletion construct is prepared such that the two target sequences are oriented in the same direction in relation to one another as they natively appear in the genome of the host cell. The gene expression cassette is cloned into the construct between the two target gene sequences to allow for expression of the exogenous gene. The gene expression cassette contains the exogenous gene, and may further include one or more regulatory sequences such as promoters or terminators operatively linked to the exogenous gene.

It is usually desirable that the deletion construct may also include a functional selection marker cassette. When a single deletion construct is used, the marker cassette resides on the vector downstream (i.e., in the 3′ direction) of the 5′ sequence from the target locus and upstream (i.e., in the 5′ direction) of the 3′ sequence from the target locus. Successful transformants will contain the selection marker cassette, which imparts to the successfully transformed cell some characteristic that provides a basis for selection.

A cell is considered to be “resistant” to a compound if it is capable of remaining viable in the presence of the substance. In some instances a resistant cell may be capable of growth and multiplication in the presence of the compound. For example, a host cell, such as a recombinant microorganism that is engineered to produce methylenemalonic acid or an intermediate is resistant to the methylenemalonic acid or intermediate if it remains viable in the presence of the methylenemalonic acid or intermediate. For example, a recombinant microorganism is resistant to methylenemalonic acid or its intermediate if it remains viable in the presence of media containing at least 1%, 3%, 5%, 6%, 7%, 8%, 9% or 10% of the methylenemalonic acid or intermediate. Test methods for determining a microorganism's resistance to compounds are well known in the art, for example the test method described in Example 1A of WO 2012/103261 and/or Example 1 provided below can be used.

A “selection marker gene” may encode for a protein needed for the survival and/or growth of the transformed cell in a selective culture medium. Typical selection marker genes encode proteins that (a) confer resistance to antibiotics or other toxins (for example, zeocin (Streptoalloteichus hindustanus ble bleomycin resistance gene), G418 (kanamycin-resistance gene of Tn903) or hygromycin (aminoglycoside antibiotic resistance gene from E. coli), (b) complement auxotrophic deficiencies of the cell (such as, for example, amino acid leucine deficiency (K. marxianus LEU 2 gene) or uracil deficiency (e.g., K. marxianus or S. cerevisiae URA3 gene)); (c) enable the cell to synthesize critical nutrients not available from simple media, or (d) confer ability for the cell to grow on a particular carbon source, (such as a MEL5 gene from S. cerevisiae, which encodes the alpha-galactosidase (mellibiase) enzyme and confers the ability to grow on melibiose as the sole carbon source). Preferred selection markers include the zeocin resistance gene, G418 resistance gene, a MEL5 gene, a URA3 gene and hygromycin resistance gene. Another preferred selection marker is an L-lactate:ferricytochrome c oxidoreductase (CYB2) gene cassette, provided that the host cell either natively lacks such a gene or that its native CYB2 gene(s) are first deleted or disrupted.

The construct may be designed so that the selection marker cassette can become spontaneously deleted as a result of a subsequent homologous recombination event. A convenient way of accomplishing this is to design the vector such that the selection marker gene cassette is flanked by direct repeat sequences. Direct repeat sequences are identical DNA sequences, native or not native to the host cell, and oriented on the construct in the same direction with respect to each other. The direct repeat sequences are advantageously about 50-1500 bp in length. It is not necessary that the direct repeat sequences encode for anything. This construct permits a homologous recombination event to occur. This event occurs with some low frequency, resulting in cells containing a deletion of the selection marker gene and one of the direct repeat sequences. It may be necessary to grow transformants for several rounds on nonselective or selective media to allow for the spontaneous homologous recombination to occur in some of the cells. Cells in which the selection marker gene has become spontaneously deleted can be selected or screened on the basis of their loss of the selection characteristic imparted by the selection marker gene, or by using PCR or Southern Analysis methods to confirm the loss of the selection marker.

In some embodiments, an exogenous gene may be inserted using DNA from two or more integration fragments, rather than a single fragment. In these embodiments, the 3′ end of one integration fragment contains a region of homology with the 5′ end of another integration fragment. One of the fragments will contain a first region of homology to the target locus and the other fragment will contain a second region of homology to the target locus. The gene cassette to be inserted can reside on either fragment, or be divided among the fragments, with a region of homology at the 3′ and 5′ ends of the respective fragments, so the entire, functional gene cassette is produced upon a crossover event. The cell is transformed with these fragments simultaneously. A selection marker may reside on any one of the fragments or may be divided between the fragments with a region of homology as described. In other embodiments, transformation from three or more constructs can be used in an analogous way to integrate exogenous genetic material.

Deletions and/or disruptions of native genes can be performed by transformation methods, by mutagenesis and/or by forced evolution methods. In mutagenesis methods cells are exposed to ultraviolet radiation or a mutagenic substance, under conditions sufficient to achieve a high kill rate (60-99.9%, preferably 90-99.9%) of the cells. Surviving cells are then plated and selected or screened for cells having the deleted or disrupted metabolic activity. Disruption or deletion of the desired native gene(s) can be confirmed through PCR or Southern analysis methods.

Cells as herein described can be cultivated to produce intermediates, methylenemalonic acid and/or corresponding esters thereof, either in the free acid form or in salt form (or both). The recombinant cell is cultured in a medium that includes at least one carbon source that can be fermented by the cell. Examples include, but are not limited to, twelve carbon sugars such as sucrose, hexose sugars such as glucose or fructose, glycan, starch, or other polymer of glucose, glucose oligomers such as maltose, maltotriose and isomaltotriose, panose, and fructose oligomers, and pentose sugars such as xylose, xylan, other oligomers of xylose, or arabinose.

The medium will typically contain, in addition to the carbon source, nutrients as required by the particular cell, including a source of nitrogen (such as amino acids, proteins, inorganic nitrogen sources such as ammonia or ammonium salts, and the like), and various vitamins, minerals and the like. In some embodiments, the cells herein described can be cultured in a chemically defined medium.

Other cultivation conditions, such as temperature, cell density, selection of substrate(s), selection of nutrients, and the like are not considered to be critical to the present technology and are generally selected to provide an economical process. Temperatures during each of the growth phase and the production phase may range from above the freezing temperature of the medium to about 50° C., although this depends to some extent on the ability of the strain to tolerate elevated temperatures. A preferred temperature, particularly during the production phase, is about 27 to 45° C.

During cultivation, aeration and agitation conditions may be selected to produce a desired oxygen uptake rate. The cultivation may be conducted aerobically, microaerobically, or anaerobically, depending on pathway requirements. For example, cultivation conditions may be selected to produce an oxygen uptake rate of around 2-25 mmol/L/hr, around 5-20 mmol/L/hr, or around 8-15 mmol/L/hr. “Oxygen uptake rate” or “OUR” as used herein refers to the volumetric rate at which oxygen is consumed during the fermentation. Inlet and outlet oxygen concentrations can be measured with exhaust gas analysis, for example by mass spectrometers. OUR can be calculated using the Direct Method described in Bioreaction Engineering Principles 2nd Edition, 2003, Kluwer Academic/Plenum Publishers, p. 449, equation I.

The cultivation may be continued until a yield of desired product on the carbon source is, for example, at least 10%, at least 20%, at least 30%, at least 40%, at least 50%, at least 60%, at least 70% or greater than 70% of the theoretical yield. The yield of product can be at least 80% or at least 90% of the theoretical yield. The concentration, or titer, of product produced in the cultivation will be a function of the yield as well as the starting concentration of the carbon source. In certain embodiments, the titer may reach at least 1, at least 3, at least 5, at least 10, at least 20, at least 30, at least 40, at least 50, or greater than 50 g/L at some point during the fermentation, and preferably at the end of the fermentation.

The term “convert” refers to the use of either chemical means or polypeptides in a reaction which changes a first intermediate to a second intermediate. The term “chemical conversion” refers to reactions that are not actively facilitated by polypeptides. The term “biological conversion” refers to reactions that are actively facilitated by polypeptides. Conversions can take place in vivo or in vitro. When biological conversions are used the polypeptides and/or cells can be immobilized on supports such as by chemical attachment on polymer supports. The conversion can be accomplished using any reactor known to one of ordinary skill in the art, for example in a batch or a continuous reactor.

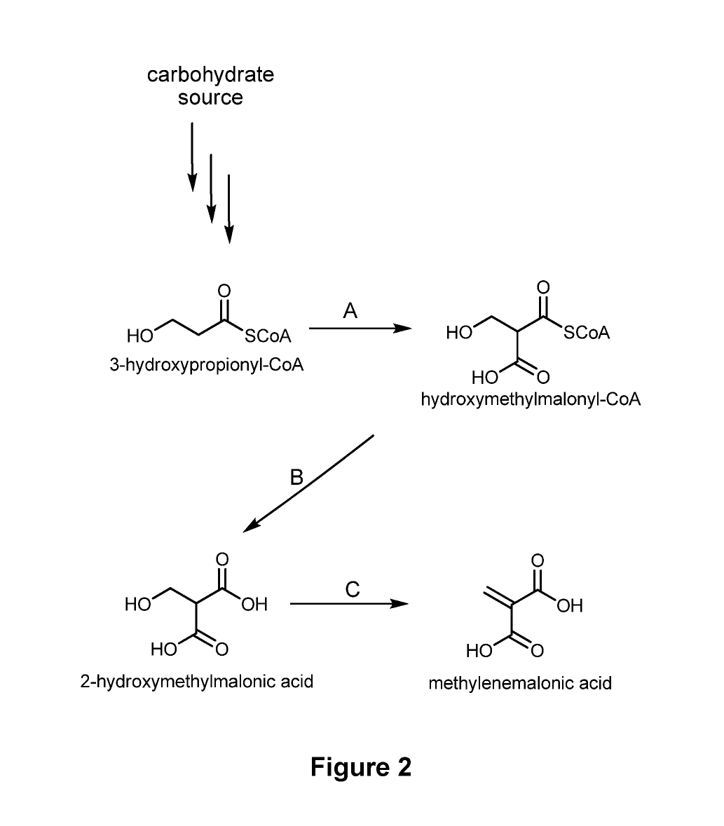

Methods are also provided that include contacting a first polypeptide with a substrate and making a first product, and then contacting the first product created with a second polypeptide and creating a second product, and then contacting the second product created with a third polypeptide and creating a third product etc. The polypeptides used to convert an intermediate to the next product or next intermediate in a pathway are described in FIGS. 2 to 6, Examples 2 to 6 and Tables 1 to 6.

The term, “compound,” as used herein is meant to include all stereoisomers, geometric isomers, tautomers, and isotopes of the structures depicted. Compounds herein identified by name or structure as particular tautomeric forms are intended to include other tautomeric forms unless otherwise specified. All compounds, salts, esters, and lactones thereof, can be found together with other substances such as water and solvents (e.g. hydrates and solvates).

The term “salt” includes any ionic form of a compound and one or more counter-ionic species (cations and/or anions). Salts also include zwitterionic compounds (i.e., a molecule containing one more cationic and anionic species, e.g., zwitterionic amino acids). Counter ions present in a salt can include any cationic, anionic, or zwitterionic species. Exemplary anions include, but are not limited to: chloride, bromide, iodide, nitrate, sulfate, bisulfate, sulfite, bisulfite, phosphate, acid phosphate, perchlorate, chlorate, chlorite, hypochlorite, periodate, iodate, iodite, hypoiodite, carbonate, bicarbonate, isonicotinate, acetate, trichloroacetate, trifluoroacetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, trifluormethansulfonate, ethanesulfonate, benzensulfonate, p-toluenesulfonate, p-trifluoromethylbenzenesulfonate, hydroxide, aluminates and borates. Exemplary cations include, but are not limited to: monovalent alkali metal cations, such as lithium, sodium, potassium, and cesium, and divalent alkaline earth metals, such as beryllium, magnesium, calcium, strontium, and barium. Also included are transition metal cations, such as gold, silver, copper and zinc, as well as non-metal cations, such as ammonium salts. A person skilled in the art will appreciate that when fully biological routes are used to produce compounds, the compound will be substantially in acid form or in salt form depending upon the pKa of the compound and the pH of the media.

An “ester” as used herein includes, as non-limiting examples, methyl esters, ethyl esters, isopropyl esters, and esters which result from the addition of a protecting group on a corresponding carboxyl moiety.

The term “unsubstituted” refers to a functional group not including substituents, for instance, an alkyl group including only carbon and hydrogen atoms. An unsubstituted group may be linear or branched.

As used herein, chemical structures which contain one or more stereocenters depicted with bold and dashed bonds (i.e., [symbol] ) are meant to indicate absolute stereochemistry of the stereocenter(s) present in the chemical structure. As used herein, bonds symbolized by a simple line do not indicate a stereo-preference. Unless otherwise indicated to the contrary, chemical structures, which include one or more stereocenters, illustrated herein without indicating absolute or relative stereochemistry encompass all possible steroisomeric forms of the compound (e.g., diastereomers, enantiomers) and mixtures thereof. Structures with a single bold or dashed line, and at least one additional simple line, encompass a single enantiomeric series of all possible diastereomers.

Compounds, as described herein, can also include all isotopes of atoms occurring in the intermediates or final compounds. Isotopes include those atoms having the same atomic number but different mass numbers. For example, isotopes of hydrogen include tritium and deuterium.

In some embodiments, the compounds described herein, or salts, esters, or lactones thereof, are substantially isolated. By “substantially isolated” is meant that the compound is at least partially or substantially separated from the environment in which it was formed or detected. Partial separation can include, for example, a composition enriched in the compounds of the presence disclosure. Substantial separation can include compositions containing at least about 50%, at least about 60%, at least about 70%, at least about 80%, at least about 90%, at least about 95%, at least about 97%, or at least about 99% by weight of the compounds herein described, or salt thereof. Methods for isolating compounds and their salts are routine in the art.

It is appreciated that certain features of the disclosure, which are, for clarity, described in the context of separate embodiments, can also be provided in combination in a single embodiment. Conversely, various features herein described which are, for conciseness, described in the context of a single embodiment, can also be provided separately or in any suitable subcombination.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about”, whether or not the term is explicitly used, unless explicitly stated otherwise. As used herein, the singular forms “a,” “an,” and “the” include plural referents unless the context clearly dictates otherwise.

### I. Engineered Pathways

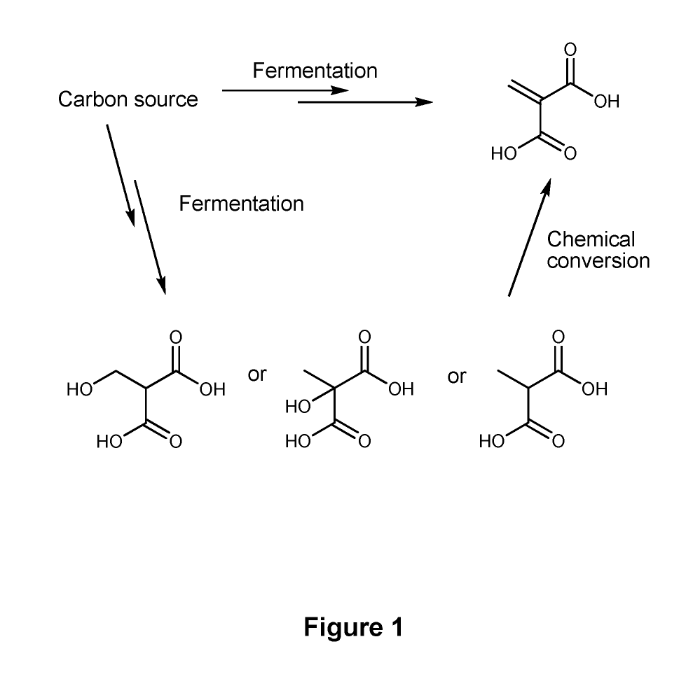

The recombinant microorganisms described herein display enzyme activities that enable them to make a non-natural amount of methylenemalonic acids or an intermediate thereof as shown in FIGS. 1 to 6, and/or a salt or corresponding ester thereof. In some instances the recombinant microorganism produces more than one type of methylenemalonic acid or intermediate thereof. The phrase “non-natural” amount refers to the fact that the recombinant microorganisms described herein produce a higher concentration of the methylenemalonic acid or intermediate thereof as compared to the host cell used as starting point for introducing the recombinant nucleic acid sequences.

One of ordinary skill in the art of metabolic engineering will appreciate that the figures provided herein describe multiple different pathways that can be used to arrive at the same methylenemalonic acid or intermediate thereof. These pathways can include enzymatic steps that rely upon an endogenous enzyme activity. Similarly, the activity of the endogenous gene can be altered through recombinant techniques to increase or decrease the endogenous transaminase activity in the host cell.

For instance, FIG. 2 shows a semi-synthetic route for making methylenemalonic acid through the 2-hydroxymethylmalonic acid intermediate. This pathway can be engineered into any host that either has been engineered to, or naturally makes, 3-hydroxymethylmalonic acid. 3-Hydroxypropionyl-CoA can be converted to hydroxymethylmalonyl-CoA using polypeptides having the enzymatic CoA carboxylase activities described in Table 1, row A. The hydroxymethylmalonyl-CoA can, in turn, be converted to 2-hydroxymethylmalonic acid using polypeptides having the enzymatic CoA hydrolase activities described in Table 1, row B. The 2-hydroxymethylmalonic acid can then be converted to methylenemalonic acid by chemical conversion (dehydration).

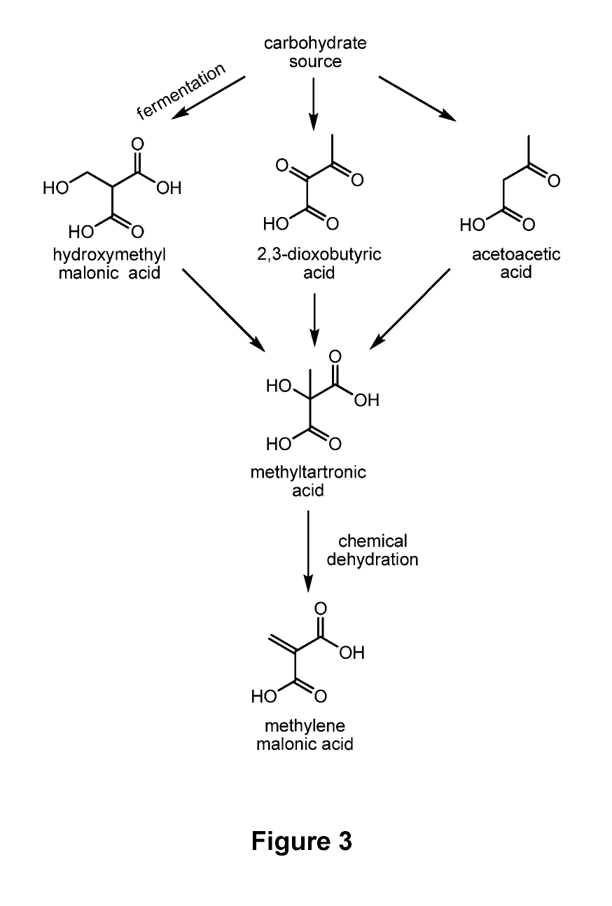

FIG. 3 shows examples of semi-synthetic routes for making methylenemalonic acid through a methyltartronic acid intermediate. This pathway can be engineered into any host that either has been engineered to, or naturally makes, 3-hydroxymethylmalonic acid, 2,3-dioxobutyric acid or acetoacetic acid. The microorganism may produce the hydroxymethylmalonic acid as described above. The hydroxymethylmalonic acid may be converted to methyltartronic acid via a dehydration-hydration reaction. Where the fermentation product is acetoacetic acid, acetoacetyl-CoA can be produced through the mevalonate pathway in cholesterol biosynthesis and in ketogenesis from acetyl-CoA in a thiolase reaction mediated by an acetyl-CoA C-acetyltransferase or acetoacetyl-CoA synthetase. The acetoacetyl-CoA can be converted to acetoacetic acid using polypeptides having enzymatic CoA hydrolase activity. The acetoacetic acid is converted to methyltartronic acid by chemical conversion.

Where the fermentation product is 2,3-dioxobutyric acid, 2,3-dihydroxybutyric acid may be produced via thiolase condensation of acetyl-CoA and glycolyl-CoA such as bktB, followed by action of 3-hydroxybutyryl-CoA reductase and a thioesterase such as phaB and tesB, respectively. The 2,3-dihydroxybutyric acid can be converted to 2,3-dioxobutyric acid using polypeptides having alcohol dehydrogenase activity. The 2,3-dioxobutyric acid may be converted to methyltartronic acid by chemical conversion (e.g. hydrolysis).

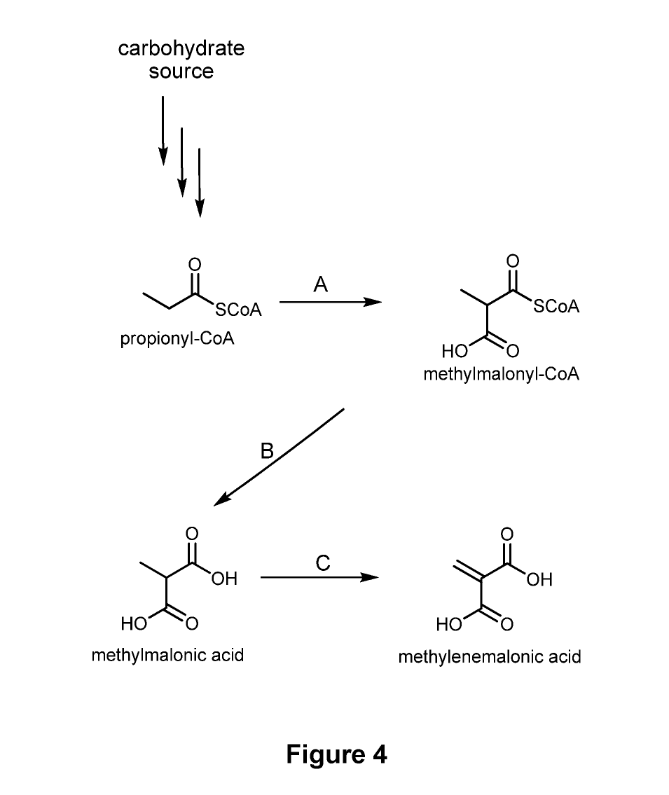

FIG. 4 shows an example of a semi-synthetic route for making methylenemalonic acid through the methylmalonic acid intermediate such as exemplified in Example 4. This pathway can be engineered into any host that either has been engineered to, or naturally makes, propionyl-CoA. The propionyl-CoA can be converted to methylmalonyl-CoA using polypeptides having the enzymatic activities described in Table 1, row A. The methylmalonyl-CoA can be converted to methylmalonic acid using polypeptides having the enzymatic activities described in Table 1, row B. The methylmalonic acid can then be converted to methylenemalonic acid by chemical conversion (dehydrogenation).

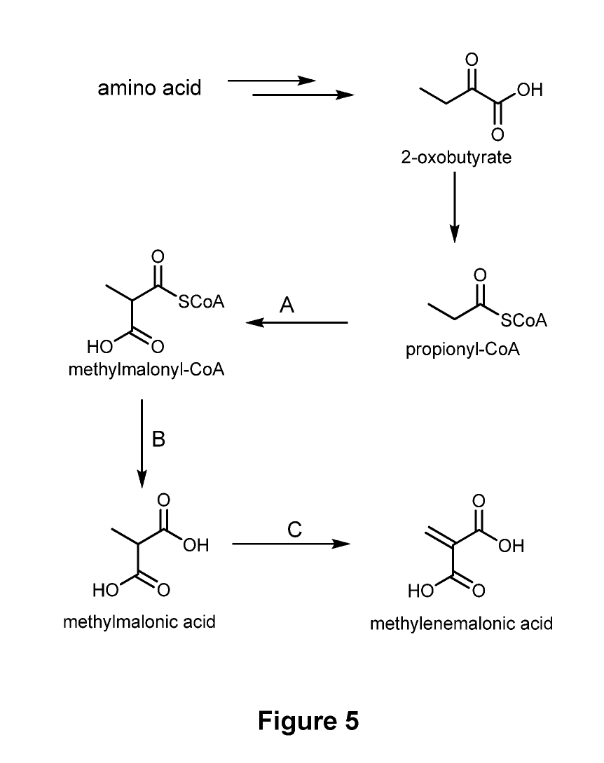

FIG. 5 shows another example of a semi-synthetic route for making methylenemalonic acid through the methylmalonic acid intermediate. This route starts from amino acids (e.g. homoserine, methionine or threonine) as carbon source. The amino acid can first be converted to 2-oxobutyrate using polypeptides having enzymatic activities such as threonine ammonia-lyase or methionine gamma-lyase described in Example 5. The 2-oxobutyrate can be converted to propionyl-CoA using polypeptides having pyruvate synthase enzymatic activities such as those described in Example 5. The propionyl-CoA can be converted to methylmalonyl-CoA, and in turn to methylmalonic acid using polypeptides as described above for FIG. 4. The methylmalonic acid can then be converted to methylenemalonic acid by chemical conversion (dehydrogenation).

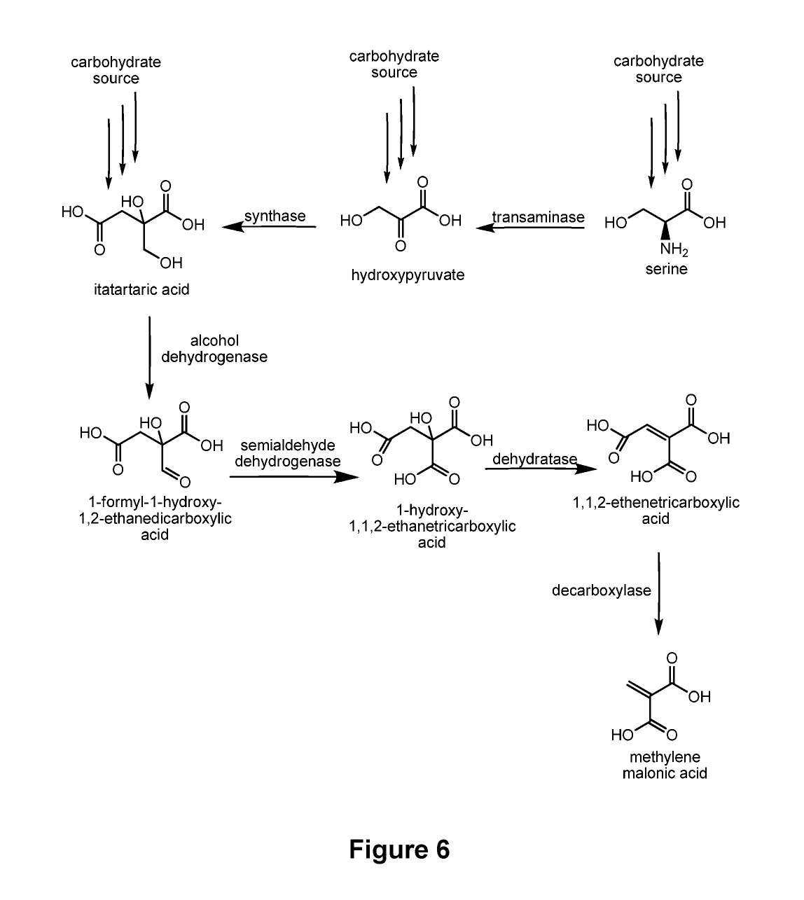

FIG. 6 shows an example of a fully or partially biological route for making methylenemalonic acid through the 1,2,2-ethylenetricarboxylic acid intermediate. This pathway can be engineered into any host that either has been engineered to, or naturally makes, serine. The serine can be converted to hydroxypyruvate using polypeptides having transaminase enzymatic activities such as those described in Table 2. The 2-hydroxypyruvate can be converted to itatartaric acid using polypeptides having synthase enzymatic activities such as those described in Tables 2, 3 and 4. The itatartaric acid obtained can be converted to 1-formyl-1-hydroxy-1,2-ethanedicarboxylic acid using polypeptides having alcohol dehydrogenase enzymatic activities such as those described in Table 2. The 1-formyl-1-hydroxy-1,2-ethanedicarboxylic acid can be converted to 1-hydroxy-1,1,2-ethanetricarboxylic acid using polypeptides having semialdehyde dehydrogenase enzymatic activities such as those described in Table 2. The 1-hydroxy-1,1,2-ethanetricarboxylic acid can be converted to 1,1,2-ethenetricarboxylic acid using polypeptides having dehydratase enzymatic activities such as those described in Tables 2 and 5. The 1,1,2-ethenetricarboxylic acid can then be converted to methylenemalonic acid using polypeptides having decarboxylase enzymatic activities such as those described in Tables 2 and 6.

In one alternative, an intermediate is produced by a biological process as described herein, isolated and converted to methylenemalonic acid via chemical conversion. For instance, the intermediate may be 2-hydroxymethylmalonic acid, 2-hydroxy-2-methylmalonic acid, methyltartronic acid, methylmalonic acid, 2-carboxymalic acid or 2-carboxymaleic acid. Examples of chemical conversion steps include, without limitation, dehydration, dehydrogenation, decarboxylation, and the like. A chemical conversion may refer to a one-step process or a multi-step process. For instance, a “dehydrogenation” conversion may also be the result of a combination of steps, e.g. hydroxylation and dehydration steps.

One of ordinary skill in the art will appreciate that the enzymes (as used herein enzymes are interchangeably referred to as polypeptides having activity) identified in the figures and elsewhere herein are exemplary enzymes and that their activities and substrate specificity can be easily tested and altered. Moreover, new enzymes having the same activities will be identified in the future and that such future discovered enzymes can be used in the described pathways.

In some examples, polypeptides having one or more point mutations that allow the substrate specificity and/or activity of the polypeptides to be modified, are used to make intermediates and products.

A variety of different carbon sources could be used to make the desired product. Examples of suitable carbon sources may include corn sugar, sucrose, glucose, xylose, glycerin, methane, methanol, acetic acid, biomass sugars, organic acids, sugar alcohols, celluloses, and/or other organic molecules. A microorganism can be engineered to utilize (or more efficiently utilize) a particular carbon source by engineering into the microorganism known enzymatic activities (e.g., to introduce transporters and/or other enzymatic activities). For example, if it is desired to produce a product from xylose, the enzymatic activities described in WO2014164410 can be introduced into the recombinant microorganism. The carbon source desired may also guide the recombinant microorganism and/or strain that is chosen to make the desired product. For example, if a particular carbon source is to be utilized, then a host strain that naturally can utilize that carbon source may be selected and engineered to produce the desired product. Further examples of different carbon sources (or carbohydrate sources) that may be used are indicated by the multiple stacked arrows shown in FIGS. 1 to 6. As one of ordinary skill in the art will appreciate, the multiple stacked arrows indicate that a variety of different enzymatic activities may be utilized by the recombinant microorganism, depending on the type of carbohydrate source.

The biosynthetic pathways described herein can be engineered into host organisms that naturally, or have already been engineered to, overproduce an intermediate in the pathway. For example, a host cell that already produces a high concentration of hydroxypropionyl-CoA, propionyl-CoA, methylmalonyl-CoA, 2-oxobutyrate, methyltartronic acid, 2-carboxymalic acid, 2-carboxymaleic acid, hydroxypyruvate, itatartaric acid, or an amino acid (e.g. threonine, homoserine or methionine) can be chosen for use as the recombinant host cell into which one or more recombinant nucleic acid sequences will be included to produce the desired methylenemalonic acid or intermediate thereof.

One of ordinary skill in the art will appreciate that regardless of the carbon source(s) used in the fermentation broth to support growth of the recombinant microorganism the economic reality is that there is a desire to maximize the carbon utilization from that carbon source(s) for product production. Generally, this is accomplished by attenuating or completely disrupting unwanted biosynthetic pathways that are otherwise native in the wild type host strain. The desired pathway will be engineered to divert carbon flow because the engineered pathway may have an increased level of enzymatic activity for a substrate that is normally found in the host cell. For example, the recombinant microorganism may display increased flux (or carbon flow) through alpha-ketoglutarate, or alternatively for an amino acid. One of ordinary skill in the art can then review which pathways cause a diversion of carbon from central metabolism up stream or prior to the branch point for the engineered pathway. These diverting pathways can then be attenuated or knocked out so that more carbon is funneled to the desired product. Examples, of pathways that can be attenuated or knocked out include pathways to products such as ethanol, acetate, glycerol and the like (see examples in WO2008116853). Other examples of activities that can be attenuated include those associated with the following enzymes: pyruvate oxidase (poxB), pyruvate-formate lyase (pflB), phosphotransacetylase (pta), acetate kinase (ackA), aldehyde dehydrogenase (aldB), alcohol dehydrogenase (adhE), alcohol dehydrogenase (adhP), methylglyoxal synthase (mgsA), and lactate dehydrogenase (IdhA).

The design of a commercially viable biosynthetic pathway should have sufficient yield of product compared to the consumed carbon source and it should also be capable of producing the product in a balanced manner. Meaning that the overall products and cofactors consumed and produced by the recombinant microorganism should result in no net surplus or deficit which would tax to host cells ability to produce the product. For example, if the overall pathway consumes acetyl CoA, an additional source of acetyl CoA may need to be engineered into the pathway. Alternatively, if an excess of a co-product occurs (e.g., acetic acid, ethanol, and/or glycerol), an appropriate mechanism for transporting the co-product or consuming the co-product should be included in the pathway.

### II. Chemical Conversion

Where methods for preparing methylenemalonic acid are semi-synthetic, one part of the process will involve fermentation of an engineered microorganism, the last or few last steps being achieved by chemical conversion, i.e. by one or more synthetic steps.

For instance, the semi-synthetic methods described herein may include the conversion of intermediates such as 2-hydroxymethylmalonic acid, 2-hydroxy-2-methylmalonic acid or methylmalonic acid, to methylenemalonic acid, and esters and/or salts thereof. The conversion from 2-hydroxymethylmalonic acid to methylenemalonic acid is a dehydration step illustrated in Scheme 1.

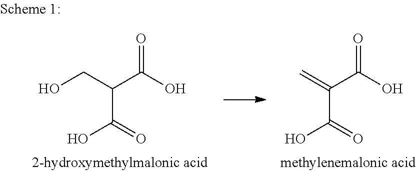

or a salt or ester thereof.

This dehydration step may be carried out for instance, by heating a solution of the 2-hydroxymethylmalonic acid and/or treating the compound with a catalyst such as silica alumina, γ-alumina, SiO₂, sulfuric acid, NaH₂PO₄-silica gel, or a mixture of phosphoric and sulfuric acid. For instance, the conditions may be similar to those described for 3-hydroxypropionic acid to acrylic acid in U.S. Pat. Nos. 7,538,247, 9,029,596, 8,338,145, and 9,181,170.

The conversion from 2-hydroxy-2-methylmalonic acid to methylenemalonic acid is a dehydration as illustrated in Scheme 2.

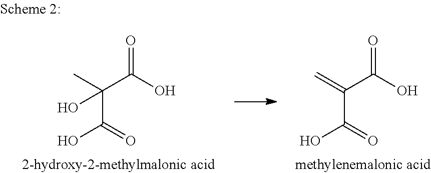

or a salt or ester thereof.

This dehydration step may be carried out for instance, by heating a solution of the 2-hydroxy-2-methylmalonic acid and/or treating the compound with a catalyst such as nickel followed by pyrolysis or reaction with bromination material, for example N-bromosuccinimide, followed by reaction with elimination material, for example triethylamine. For instance, the conditions may be similar to those described for the conversion of lactic acid to acrylic acid in US2012078004A1 and U.S. Pat. No. 9,260,550B1.

The conversion from methylmalonic acid to methylenemalonic acid may be a dehydrogenation step illustrated as in Scheme 3.

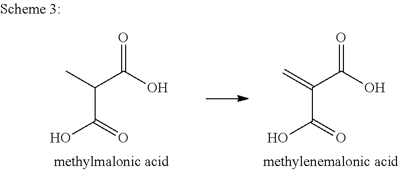

or a salt or ester thereof.

This dehydrogenation step may be carried out for instance, by heating a solution of methylmalonic acid especially in the presence of O₂, steam, and N₂ and/or treating a solution of the methylmalonic acid with a catalyst such as Vn, Mo, P, As, Cs, ZrS, (VO)_{1.5}Cu_{0.5}PMo₁₁VO₄₀, or Fe₂(PO₃OH)P₂O₇. Conditions may be similar to those described for converting isobutyric acid to acrylic acid in U.S. Pat. Nos. 5,618,974, 5,335,954, and Bonnet et al, Journal of Catalysis, 158.1 (1996): 128-141.

Preparation of the compounds as described herein may involve the protection and deprotection of various chemical groups. The need for protection and deprotection, and the selection of appropriate protecting groups, can be readily determined by one skilled in the art. In the chemical conversions described above, protecting groups may also be used, for instance, on carboxyl groups. For this purpose, the protecting group may include any suitable carboxyl protecting group such as, but not limited to, esters, amides, or hydrazine protecting groups. The protecting group may be the same or different in each occurrence.

In particular, an ester protecting group may include methyl, methoxy methyl (MOM), benzyloxymethyl (BOM), methoxyethoxymethyl (MEM), 2-(trimethylsilyl)ethoxymethyl (SEM), methylthiomethyl (MTM), phenylthiomethyl (PTM), azidomethyl, cyanomethyl, 2,2-dichloro-1,1-difluoroethyl, 2-chloroethyl, 2-bromoethyl, tetrahydropyranyl (THP), 1-ethoxyethyl (EE), phenacyl, 4-bromophenacyl, cyclopropylmethyl, allyl, propargyl, isopropyl, cyclohexyl, t-butyl, benzyl, 2,6-dimethyl benzyl, 4-methoxybenzyl (MPM-OAr), o-nitrobenzyl, 2,6-dichlorobenzyl, 3,4-dichlorobenzyl, 4-(dimethylamino)carbonylbenzyl, 4-methylsulfinylbenzyl (Msib), 9-anthrylmethyl, 4-picolyl, heptafluoro-p-tolyl, tetrafluoro-4-pyridyl, trimethylsilyl (TMS), t-butyldimethylsilyl (TBDMS), t-butyldiphenylsilyl (TBDPS), and triisopropylsilyl (TIPS) protecting groups.

The amide and hydrazine protecting groups may include N,N-dimethylamide, N-7-nitroindoylamide, hydrazide, N-phenylhydrazide, and N,N′-diisopropylhydrazide.

In some embodiments, a hydroxyl group may be protected. For this purpose, the protecting group may include any suitable hydroxyl protecting group including, but not limited to, ether, ester, carbonate, or sulfonate protecting groups. Each occurrence of the protecting group may be the same or different.

In particular, the ether protecting group may include methyl, methoxy methyl (MOM), benzyloxymethyl (BOM), pivaloyloxymethyl (POM), methoxyethoxymethyl (MEM), 2-(trimethylsilyl)ethoxymethyl (SEM), methylthiomethyl (MTM), phenylthiomethyl (PTM), azidomethyl, cyanomethyl, 2,2-dichloro-1,1-difluoroethyl, 2-chloroethyl, 2-bromoethyl, tetrahydropyranyl (THP), 1-ethoxyethyl (EE), phenacyl, 4-bromophenacyl, cyclopropylmethyl, allyl, propargyl, isopropyl, cyclohexyl, t-butyl, benzyl, 2,6-dimethylbenzyl, 4-methoxybenzyl (MPM-OAr), o-nitrobenzyl, 2,6-dichlorobenzyl, 3,4-dichlorobenzyl, 4-(dimethylamino)carbonylbenzyl, 4-methylsulfinylbenzyl (Msib), 9-anthrylemethyl, 4-picolyl, heptafluoro-p-tolyl, tetrafluoro-4-pyridyl, trimethylsilyl (TMS), t-butyldimethylsilyl (TBDMS), t-butyldiphenylsilyl (TBDPS), and triisopropylsilyl (TIPS) protecting groups.

The ester protecting group may include acetoxy (OAc), formate, levulinate, pivaloate, benzoate, and 9-fluorenecarboxylate. In one embodiment, the ester protecting group is an acetoxy group.

The carbonate protecting group may include aryl or methyl carbonate, 1-adamantyl carbonate (Adoc-O—), t-butyl carbonate (BOC—O—), 4-methylsulfinylbenzyl carbonate (Msz-O—), 2,4-dimethylpent-3-yl carbonate (Doc-O—), 2,2,2-trichloroethyl carbonate, vinyl carbonate, benzyl carbonate, and aryl carbamate.

The sulfonate protecting groups may include methanesulfonate, toluenesulfonate, and 2-formylbenzenesulfonate.

The chemistry of protecting groups, including protection and deprotection conditions, can be found, for example, in Protecting Group Chemistry, 1^{st} Ed., Oxford University Press, 2000; March's Advanced Organic chemistry: Reactions, Mechanisms, and Structure, 5^{th} Ed., Wiley-Interscience Publication, 2001; and Peturssion, S. et al., “Protecting Groups in Carbohydrate Chemistry,” J. Chem. Educ., 74(11), 1297 (1997) (each being incorporated herein by reference in their entirety).

A metal catalyst as used herein can include any suitable metal catalyst. For example, a suitable metal catalyst would include one that can facilitate the conversion of one or more of 2-hydroxymethylmalonic acid, methyltartronic acid, or methylmalonic acid, or salts or esters thereof, to methylenemalonic acid, a salt or ester thereof.

In some embodiments, a suitable metal catalyst for the present methods is a heterogeneous (or solid) catalyst. The metal catalyst (e.g., a heterogeneous catalyst) can be supported on at least one catalyst support (referred to herein as “supported metal catalyst”). When used, at least one support for a metal catalyst can be any solid substance that is inert under the reaction conditions including, but not limited to, oxides such as silica, alumina and titania, compounds thereof or combinations thereof; barium sulfate; zirconia; carbons (e.g., acid washed carbon); and combinations thereof. Acid washed carbon is a carbon that has been washed with an acid, such as nitric acid, sulfuric acid or acetic acid, to remove impurities. The support can be in the form of powders, granules, pellets, or the like. The supported metal catalyst can be prepared by depositing the metal catalyst on the support by any number of methods well known to those skilled in the art, such as spraying, soaking or physical mixing, followed by drying, calcination, and if necessary, activation through methods such as heating, reduction, and/or oxidation. In some embodiments, activation of the catalyst can be performed in the presence of hydrogen gas. For example, the activation can be performed under hydrogen flow or pressure (e.g., a hydrogen pressure of about 200 psi). In some embodiments, the metal catalyst is activated at a temperature of about 100° C. to about 500° C. (e.g., about 100° C. to about 500° C.).

In some embodiments, the loading of the at least one metal catalyst on the at least one support is from about 0.1 weight percent to about 20 weight percent based on the combined weights of the at least one acid catalyst plus the at least one support. For example, the loading of the at least one metal catalyst on the at least one support can be about 5% by weight.

A metal catalyst can include a metal selected from nickel, palladium, platinum, copper, zinc, rhodium, ruthenium, bismuth, iron, cobalt, osmium, iridium, vanadium, and combinations of two or more thereof. In some embodiments, the metal catalyst comprises copper or platinum. For example, the metal catalyst can comprise platinum.

A chemical promoter can be used to increase the activity of the catalyst. The promoter can be incorporated into the catalyst during any step in the chemical processing of the catalyst constituent. The chemical promoter generally enhances the physical or chemical function of the catalyst agent, but can also be added to retard undesirable side reactions. Suitable promoters include, for example, sulfur (e.g., sulfide) and phosphorous (e.g., phosphate). In some embodiments, the promoter comprises sulfur.

Non-limiting examples of suitable metal catalysts as described herein include nickel catalysts (e.g. Raney® Nickel, W. R. Grace), copper catalysts (e.g. Cu-0860 and Cu-0865 from BASF, Cu/Zn/Al MeOH unreduced), palladium catalysts (e.g. 10% Pd/C, 5% Pd/C, 5% Pd(S)/C, 5% Pd/Al₂O₃, 5% Pd/CaCO₃, 5% Pd(Pb)/CaCO₃, 5% Pd/BaSO₄, 5% Pd/CaCO₃, 4% Pd-1% Pt/C, 4.5% Pd-0.5% Rh/C, 0.6% Pd/C unreduced, 20% Pd/C (Pearlman's catalyst) unreduced), platinum catalysts (e.g. 3% Pt/C, 5% Pt/C, 5% Pt(Bi)/C, 5% Pt(S)/C, 5% Pt/Al₂O₃, 1% Pt-2% V/C), rhodium catalysts (e.g. 5% Rh/C, 5% Rh/Al₂O₃), and ruthenium catalysts (e.g. 5% Ru/C, 5% Ru/Al₂O₃, 5% Ru-0.25% Pd/C).

Temperature, solvent, catalyst, reactor configuration, pressure and mixing rate are all parameters that can affect the conversions described herein. The relationships among these parameters may be adjusted to effect the desired conversion, reaction rate, and selectivity in the reaction of the process.

In some embodiments, the methods provided herein are performed at temperatures from about 25° C. to about 350° C. For example, the methods can be performed at a temperature of at least about 100° C. In some embodiments, a method provided herein is performed at a temperature of about 100° C. to about 200° C. For example, a method can be performed at a temperature of about 150° C. to about 180° C.

The methods described herein may be performed neat, in water or in the presence of an organic solvent. In some embodiments, the reaction solvent comprises water. Exemplary organic solvents include hydrocarbons, ethers, and alcohols. In some embodiments, alcohols can be used, for example, lower alkanols, such as methanol, ethanol and isopropanol. The reaction solvent can also be a mixture of two or more solvents. For example, the solvent can be a mixture of water and a lower alcohol.

The methods provided herein can be performed under inert atmosphere (e.g., N₂ and Ar). In some embodiments, the methods provided herein are performed under nitrogen. For example, the methods can be performed under a nitrogen pressure of about 20 psi to about 1000 psi. In some embodiments, a method as described herein is performed under a nitrogen pressure of about 200 psi.

In some embodiments, additional reactants can be added to the methods described herein. For example, a base such as sodium hydroxide can be added to the reaction.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS) or thin layer chromatography (TLC). Compounds can be purified by those skilled in the art by a variety of methods, including high performance liquid chromatography (HPLC) (K. F. Blom, et al., J. Combi. Chem. 6(6) (2004), which is incorporated herein by reference in its entirety) and normal phase silica chromatography.

### EXAMPLES

The following examples are provided to illustrate the present technology, but are not intended to limit the scope thereof. All parts and percentages are by weight unless otherwise indicated.

### Example 1—Cells Resistant to Intermediates

Potential hosts for the described pathways to methylenemalonic acid are identified by determining the tolerance to the fermentation products as shown in FIG. 1. Individual compounds are selected to assess tolerance of bacterial and eukaryotic hosts.

Both bacterial and eukaryotic strains are tested for tolerance to the selected compounds using their individual optimal conditions. E. coli is tested at pH 8 and 30° C., grown in standard LB media consisting of 10 g/L bacto-tryptone, 5 g/L yeast extract, 10 g/L NaCl, and 20 g/L dextrose with addition of 20 g/L glucose. S. cerevisiae and K. marxianus are grown in buffered defined dextrose media consisting of 50 g/L dextrose, 5 g/L yeast extract, and 40 mL/L 25×DM salts. The 25×DM salt stock solution contains 125 g/L ammonium sulfate, 12.5 g/L magnesium sulfate heptahydrate, 75 g/L potassium phosphate monobasic, and 787.5 g/L water.

Time points are taken over a period of at least 8 hours and up to 24 hours to calculate the rate of growth. Specific growth rate is determined by plotting the natural logarithm of cell number against time. Tolerance is determined by growth rate of cells in the presence of the compound as compared to in the absence of the compound.

Tolerance studies to an analog of 2-hydroxymethylmalonic acid were performed using alpha-hydroxymethyl-3-hydroxypropionic acid having the formula:

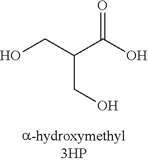

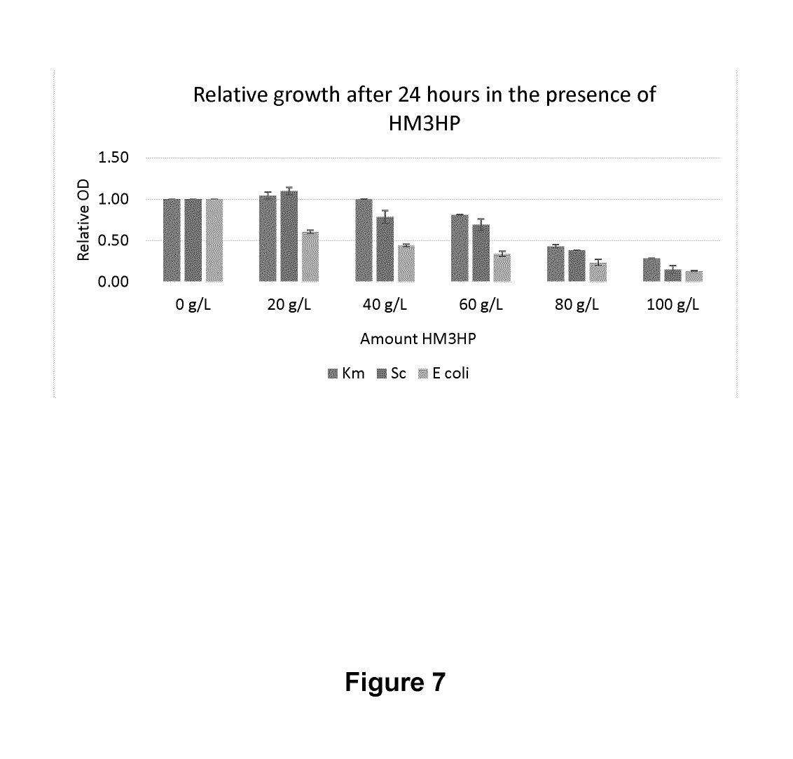

FIG. 7 presents the relative growth of host organisms after 24 hours in the presence of alpha-hydroxymethyl-3-hydroxypropionic acid. These results suggest a relative tolerance as follows K. marxianus (Km)>S. cerevisiae (Sc)>E. coli.

### Example 2—Construction of Recombinant Microorganism for the Production of 2-Hydroxymethylmalonic Acid Utilizing a 3-Hydroxypropionyl-CoA Overproducing Microorganism

The microorganism used for producing 2-hydroxymethylmalonic acid can be selected from fungi, including yeast and filamentous fungi, as well as bacteria. The microorganism engineered to express pathways described in Kumar et al, 2013 may be used as a starting 3-hydroxypropionyl-CoA (3HP-CoA) overproducing strain for subsequence genetic engineering steps. In one example, glucose is converted to 3HP via the intermediates acetyl-CoA and malonyl-CoA. In another embodiment, glucose is converted to 3HP via the intermediates pyruvate or phosphoenolpyruvate, oxaloacetate, aspartate, beta-alanine, and 3-oxopropionate. In another embodiment, glucose is converted to 3HP via intermediates alpha-alanine, beta-alanine, and 3-oxopropionate. In cases where 3HP is the product, 3HP is converted to 3HP-CoA using a CoA transferase such as 3-hydroxypropionyl-CoA synthetase from Metallosphaera sedula or acetyl-CoA transferase from E. coli (Jenkins and Nunn, 1986). In another example, 3HP is converted to 3HP-CoA using an acetyl-CoA synthetase from E. coli (Kumari et al, 1995) or yeast (Satyanarayana and Klein, 1973).

In one embodiment, glucose is converted to 3HP-CoA via the intermediates pyruvate, lactate, lactoyl-CoA, and acryloyl-CoA. In another embodiment, glucose is converted to 3HP-CoA via the intermediates pyruvate or phosphoenolpyruvate, succinate, propionyl-CoA, and acryloyl-CoA. In another embodiment, glucose is converted to 3HP-CoA via the intermediates pyruvate or phosphoenolpyruvate, oxaloacetate, aspartate, beta-alanine, beta-alanyl-CoA, and acryloyl-CoA. In another embodiment, glucose is converted to 3HP-CoA via the intermediates pyruvate, alpha-alanine, beta-alanine, beta-alanyl-CoA, and acryloyl-CoA.

The microorganism expresses all enzymes necessary to convert 3HP-CoA to 2-hydroxymethyl malonic acid. The DNA fragments encoding CoA carboxylase (FIG. 2, step A) and CoA hydrolase (FIG. 2, step B) are cloned into an expression vector. The resulting plasmid that successfully transcribes all pathway genes is transformed into a recombinant microorganism that produces 3HP-CoA as described above.

Examples of enzymes and their corresponding references are shown in Table 1 and described in the accompanying text below. In one example, the CoA carboxylase (step A) is propionyl-CoA carboxylase from Ruegeria pomeroyi (RpPCC, accession: 3N6R_K), the CoA hydrolase is thioesterase from E. coli, for example yciA. The resulting plasmid that successfully transcribes all pathway genes is transformed into a microorganism overproducing 3-hydroxypropionyl-CoA. The microorganism overproducing 3-hydroxypropionyl-CoA is described above in this example and reviewed in Kumar et al., 2013. The microorganism may be bacterial or eukaryotic (e.g., a yeast or fungus). Examples of hosts may include E. coli, Klebsiella pneumonia, Pseudomonas dentrificans, and yeast strains including S. cerevisiae.

Additionally, expression of a DNA fragment encoding a transporter may improve production of hydroxymethylmalonic acid. For example, the transporter gene may be selected from malic acid transport genes, tehA from E. coli (UNIPROT E0IVN4), mae1 from S. pombe (Saayman et al, 2000), and ykxJ from Bacillus subtilis (Krom et al, 2001), or homologs thereof.
Krom, Aardema, and Lolkema. Bacillus subtilis YxkJ is a secondary transporter of the 2-hydroxycarboxylate transporter family that transports L-malate and citrate. J Bacteriol, 2001 October; 183(20):5862-9.
Saayman, van Vuuren, van Zyl, and Viljoen-Bloom. Differential uptake of fumarate by Candida utilis and Schizosaccharaomyces pombe. Appl Microbiol Biotechnol, 2000. 54: 792-798.

TABLE 1
Enzymes and references for the pathway
to 2-hydroxymethylmalonic acid

| | Enzyme | | | |
|---|---|---|---|---|
| | category | Enzyme name | Organism | Reference |
| A | CoA | propionyl-CoA | Ruegeria pomeroyi | Huang et |
| | carboxylase | carboxylase | | al, 2010 |
| | | propionyl-CoA | Homo sapients | Jiang et |
| | | carboxylase | | al, 2005 |
| | | propionyl-CoA | Streptomyces | Arabolaza |
| | | carboxylase | coelicolor | et al, 2010. |
| | | acetyl-CoA/ | Metallosphaera | Hugler et |
| | | propionyl-CoA | sedula | al, 2003. |
| | | carboxylase | | |
| B | CoA | Thioesterase | E. coli | Bonner and |
| | hydrolase | (tesA) | | Bloch, 1972. |
| | | Thioesterase | E. coli | Naggert et |
| | | (tesB) | | al, 1991. |
| | | Thioesterase | E. coli | Zhuang et |
| | | (yciA) | | al, 2008 |
| | | methylmalonyl- | Rattus norvegicus | Kovachy et |
| | | CoA hydrolase | | al, 1983. |

Carboxylase Assay

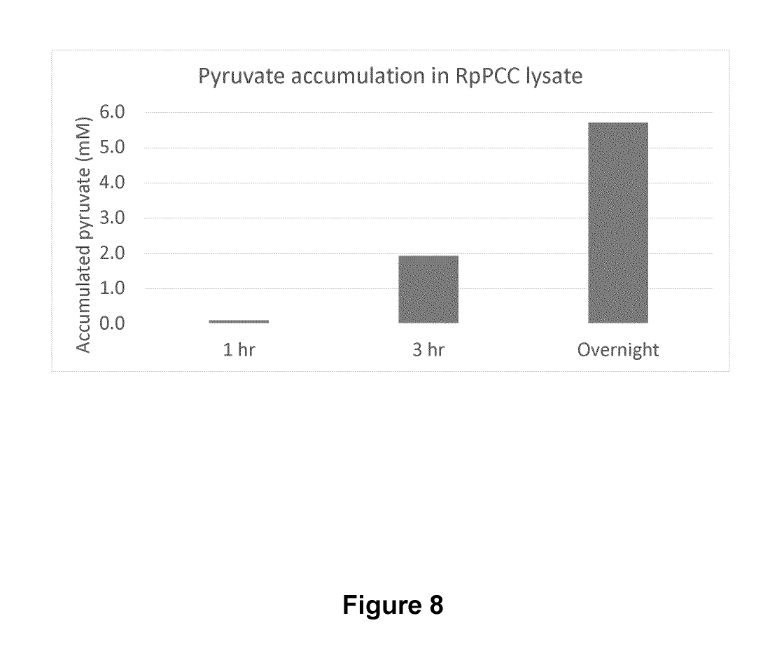

The amount of 3HP-CoA converted to 2-hydroxymethylmalonyl-CoA was measured using a coupled reaction resulting in pyruvate accumulation. E. coli cells were transformed with either empty vector (ptrc) or propionyl-CoA carboxylase from Rugeria pomeroyi (RpPCC). Cells were lysed using mechanical disruption with a BeadBeater (BopSpec products, Bartlesville, Okla.) using the manufacturer's instructions. The cell lysate was partially clarified by centrifugation (14,000 G for 5 minutes). Protein concentrations of the resulting clarified lysates were measured via BioRad total Protein assay using the manufacturer's instructions. Lysates were normalized by protein concentration with 100 mM potassium phosphate buffer, pH 7.6. The pyruvate-coupled carboxylase reaction assays contained 100 mM potassium phosphate buffer (pH 7.6), 5 μL of pyruvate kinase (2.5 units per μL), 5 mM phosphoenolpyruvate 0.3 mg/mL BSA, 5 mM MsCl₂, 50 mM NaHCO₃, 5 mM ATP, and 5 mM 3HP-CoA substrate. The reaction was started with 25 μL of lysate added to the reaction mix to reach a total volume of 100 μL. Pyruvate accumulation was assessed via HPLC. The lysate expressing RpPCC accumulated pyruvate over time indicated carboxylase of 3HP-CoA to result in 2-hydroxymethylmalonyl-CoA (FIG. 8).

CoA Hydrolase Assay

E. coli optimized genes encoding CoA hydrolases are synthesized and cloned into pTrcHisA (Life Technologies (formerly Invitrogen)). Alternatively, E coli CoA hydrolases were amplified from E. coli genome via PCR and cloned into a pTrcHisA expression vector. CoA hydrolase genes tested are found in Table 1, row B. Plasmids containing the optimized synthase genes were transformed into BL21 E. coli cells. Empty plasmid pTrcHisA was also transformed as a negative control. For expression and characterization experiments, shake flasks containing 40 mL TB were inoculated at 5% from overnight cultures. Flasks were incubated at 30° C. at 250 rpm shaking for 2 hours, then protein production was induced with 0.2 mM IPTG and incubated for 4 more hours at 30° C. while shaking. Cells were harvested by centrifugation and pellets stored at −80° C.

Activity of synthase candidates was assessed with an in vitro assay using DTNB (5,5′-dithiobis(2-nitrobenzoic acid)) as an indicator. The enzyme activity was tested using either no substrate, 3HP-CoA, or hydroxymethyl malonyl-CoA (HMMCoA) as the substrate. The DTNB interacts with free thio groups created by the condensation of acetyl-CoA and the substrate present. The substrate, HMMcoA, was synthesized using the carboxylase reaction described above. To remove protein and cell debris, the carboxylation reaction product was transferred to the 10 kDa protein spin column (Millipore) and centrifuged at 14,000 G for 10 minutes; the flowthrough was retained and used as HMMCoA substrate.

Cells were lysed using mechanical disruption using a BeadBeater (BopSpec products, Bartlesville, Okla.) following the manufacturer's instructions. The cell lysate was partially clarified by centrifugation (14,000 G for 5 minutes). Protein concentrations of the resulting clarified lysates were measured via BioRad total Protein assay using the manufacturer's instructions. Lysates were normalized by protein concentration in 100 mM potassium phosphate buffer, pH 7.6, to 5 μg/μL. The normalized lysates were diluted 1 to 20 in 100 mM Tris buffer. 20 μL of the diluted normalized lysate was added to each well for the 96-well plate assay. Each condition was performed in triplicate.

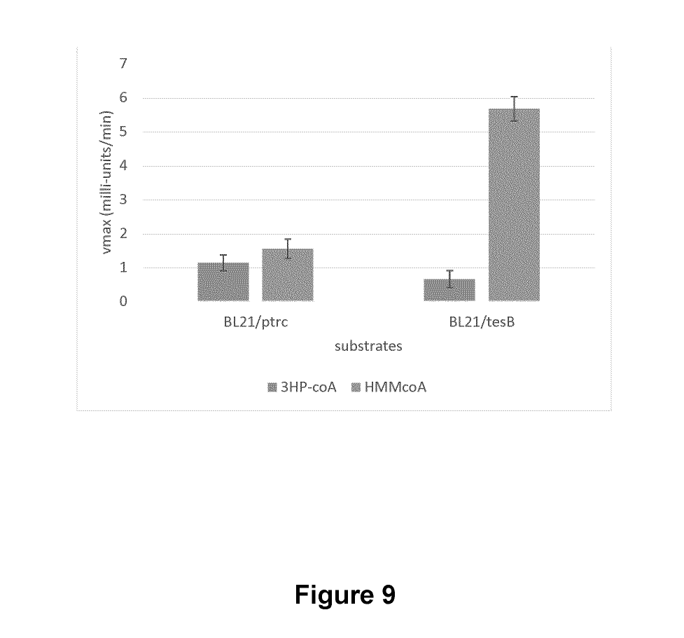

The reaction mixture contained 100 mM potassium phosphate buffer at pH 7.6, 0.125 mM CoA substrate, and 0.04 mg/mL DTNB. To start the reaction, 180 μL of reaction mix was added to each well already containing 20 μL lysate. The reactions in these microplates were monitored at 412 nm. Readings were taken every 9 seconds for 10 minutes and the data was used to calculate activities of each enzyme. Hydrolase activity was observed when free CoA concentration increases with hydroxymethylmalonyl-CoA as the substrate, as compared to cells containing the empty vector. Background absorbance, which is measured by the same reaction with no substrate present, is subtracted. The TesB expressing lysate showed activity with the carboxylated product, HMMcoA, but little to no activity with the substrate 3HP-coA (see FIG. 9).
Arabolaza, Shillito, Lin, Dicovich, Melgar, Pham, Amick, Gramajo, and Tsai. Crystal structures and mutational analyses of acyl-CoA carboxylase B subunit of Streptomyces coelicolor. Biochemistry, 2010. 49(34): 7367-7376.
Bonner and Bloch. Purification and properties of fatty acyl thioeserase I from Escherichia coli. J Biol Chem, 1972. Vol 247, No, 10, p. 8123-8133.
Huang, et al. Crystal structure of the alpha(6)beta(6) holoenzyme of propionyl-coenzyme A carboxylase. Nature 2010 Aug. 19; 466(7309):1001-1005.
Hugler, Krieger, Jahn, and Fuchs. Characterization of acetyl-CoA/propionyl-CoA carboxylase in Metallosphaera sedula. Eur J Biochem 2003. 270, 736-744.
Jenkins and Nunn. Genetic and molecular characterization of the genes involved in short-chain fatty acid degradation in Escerichia coli: the ato system. J Bacteriology, January 1987. Vol 169, No 1, p. 42-52.
Jiang, Rao, Yee, and Kraus. Characterization of Four Variant Forms of Human Propionyl-CoA carboxylase expressed in Escherichia coli. J of Biol Chem, 2005. Vol 280, No. 30.
Kovachy, Copley, and Allen. Recognition, isolation, and characterization of rat liver D-methylmalonyl coenzyme A hydrolase. J Biol Chem, 1983. Vol 25, No. 18: 11415-11421.
Kumr, Ashok, and Park. Recent advances in biological production of 3-hydroxypropionic acid. Biotechnology advances, 2013. 31, p. 945-961.
Kumari, Tishel, Eisenbach, and Wolfe. Cloning, characterization, and functional expression of acs, the gene which encodes acetyl coenzyme A synthetase in Escherichia coli. J Bacteriology, May 1995. Vol 177, No 10, p. 2878-2886.
Naggert, Narasimhan, DeVeaux, Cho, Randhawa, Cronan, Green, and Smith. Cloning, sequencing, and characterization of Escherichia coli Thioesterase II. J Biol chem, 1991. Vol 266, No. 17, pp 11044-11050.
Satyanarayana and Klein. Studies on acetyl-coenzyme A synthetase of yeast: inhibition by long-chain acyl-coenzyme A esters. J Bacteriology, August 1973. Vol 115, No 2, pp. 600-606.
Zhuang, Song, Zhao, Li, Cao, Eisenstein, Herzberg, and Dunaway-Mariano. Divergence of function in the hot dog fold enzyme superfamily: the bacterial thioesterase YciA. Biochemistry, 2008; 17(9):2789-96.

### Example 3—Construction of Recombinant Microorganism for Production of Methyltartronic Acid

The microorganism used for production of methyltartronic acid can be selected from fungi, including yeast and filamentous fungi as well as bacteria. More than one fermentation product may be converted to methyltartronic acid (FIG. 3).

The microorganism may produce the fermentation product 2-hydroxymethylmalonic acid as described in Example 2. The fermentation product 2-hydroxymethylmalonic acid is converted to methyltartronic acid via a dehydration-hydration reaction.

The microorganism may produce the fermentation product acetoacetic acid. Acetoacetyl-CoA is produced in nature as part of the mevalonate pathway in cholesterol biosynthesis and in ketogenesis in the liver. It is created from acetyl-CoA in a thiolase reaction mediated by acetyl-CoA C-acetyltransferase (EC 2.3.1.9) or acetoacetyl-CoA synthetase (EC 2.3.1.194). The addition of a CoA hydrolase converts acetoacetyl-CoA to the final fermentation product acetoacetic acid. The fermentation product acetoacetic acid is converted to methyltartronic acid via the method described in Gowal 1985.

The microorganism may produce the fermentation product 2,3-dioxobutyric acid. In another embodiment, the product 2,3-dihydroxybutyric acid may be produced as described in Martin et al, 2013 via thiolase condensation of acetyl-CoA and glycolyl-CoA such as bktB, followed by action of 3-hydroxybutyryl-CoA reductase and a thioesterase such as phaB and tesB, respectively. Subsequent oxidation by alcohol dehydrogenase enzyme or enzymes yield the final fermentation product 2,3-dioxobutyric acid. The fermentation product 2,3-dioxobutyric acid may be converted to methyltartronic acid via a hydrolysis reaction, for example using conditions as described in Davis et al, 1953.
Davis, et al. C14 tracer studies in the rearrangements of unsymmetrical a-diketones. IV. Ethyl a,b-dioxobutyrate to methyltartronic acid. Journal of American Chem Society, 1953. 75, 3304-5.
Gowal et al. Reductones and tricarbonyl compounds, part, 31. Nucleopile 1,2-shifts of alkoxycarbonyl and carboxylate groups in the benzlic-acid type rearrangement of α,β-dioxobutyric esters. Helvetica Chimica Acta, 1985. 68(1), p. 173-80.
Martin, Dhamankar, Tseng, Sheppard, Reisch, and Prather. A platform pathway for production of 3-hydroxyacids as value-added biochemical—a biosynthetic route to 3-hydroxy-γ-butyrolactone. Nature Communications, 2013. 4:1414, 1-10.

### Example 4—Construction of Recombinant Microorganism for Production of Methylmalonic Acid

The microorganism used for producing hydroxymethylmalonic acid can be selected from fungi, including yeast and filamentous fungi, as well as bacteria. The microorganism expresses all enzymes necessary to convert propionyl-CoA to methylmalonic acid. The DNA fragments encoding CoA carboxylase (FIG. 4, step A) and CoA hydrolase (FIG. 4, step B) are cloned into an expression vector. The resulting plasmid that successfully transcribes all pathway genes is transformed into a recombinant microorganism that produces propionyl-CoA as described in Chen et al, 2011. In one example, propionyl-CoA production is increased by overexpression of threonine deaminase (Chen et al, 2011).
Chen, Wang, Wei, Liant, and Qi. Production in Escherichia coli of poly(3-hydroxybutyrate-co-3-hydroxyvalerate) with differing monomer compositions from unrelated carbon sources. Applied and Environmental Microbiology, July 2011. pp. 4886-4893.

Examples of enzymes and their corresponding references are shown in Table 1 and are described in the accompanying text below. In one example, the CoA carboxylase (step A) is propionyl-CoA carboxylase from Rugeria pomeroyi (RpPCC, accession: 3N6R_K), the CoA hydrolase is a thioesterase from E. coli, yciA. The resulting plasmid that successfully transcribes all pathway genes is transformed into a recombinant microorganism that produces propionyl-CoA as described in Chen et al, 2011. In one embodiment, propionyl-CoA production is increased by overexpression of threonine deaminase (Chen et al, 2011). The microorganism may be bacterial or eukaryotic. In some implementations, the host may be a yeast or fungus. Non-limiting examples of hosts may include E. coli, Klebsiella pneumonia, Pseudomonas dentrificans, Propionibacterium freudenreichii, Propionibacterium shermanii, and yeast strains including S. cerevisiae. Assays to demonstrate activity of CoA carboxylase and CoA hydrolase enzymes are described in Example 2.

Additionally, expression of a DNA fragment encoding a methylmalonic acid transporter improves production of methylmalonic acid. For example, the transporter gene may be selected from malic acid transport genes, tehA from E. coli (UNIPROT E0IVN4), mael from S. pombe (Saayman et al, 2000), and yxk from Bacillus subtilis (Krom et al, 2001), or homologs thereof.
Krom, Aardema, and Lolkema. Bacillus subtilis YxkJ is a secondary transporter of the 2-hydroxycarboxylate transporter family that transports L-malate and citrate. J Bacteriol, 2001 October; 183(20):5862-9.
Saayman, van Vuuren, van Zyl, and Viljoen-Bloom. Differential uptake of fumarate by Candida utilis and Schizosaccharaomyces pombe. Appl Microbiol Biotechnol, 2000. 54: 792-798.

### Example 5—Construction of Recombinant Microorganism for Production of Methylmalonic Acid Utilizing an Amino Acid Overproducing Microorganism

The microorganism used for the production of methylmalonic acid can be selected from fungi, including yeast and filamentous fungi as well as bacteria. The microorganisms described in Adrio and Demain et al., 2010 can be used as a starting threonine, homoserine, or methionine overproducing strain for subsequent genetic engineering steps. In instances were bacterial production is desired, E. coli or Serratia marcencens can be used as a starting strain for subsequent genetic engineering steps. Similarly, the microorganism described in Ramos and Calderon can be used as a starting strain for subsequent genetic engineering steps in instances were eukaryotic production is desired.
Adrio and Demian. Recombinant organisms for production of industrial production. Bioengineered Bugs, March/April 2010, 1:2, 116-131.
Ramos and Calderon. Overproduction of threonine by Saccharomyces cerevisiae mutants resistant to hydroxynorvaline. App and Environ Microb, May 1992, p. 1677-1682.

In addition to all enzymes described in Example 4, the host organism overproduces an amino acid. In one embodiment the organism overexpresses threonine or homoserine. In this example threonine or homoserine is converted to 2-oxobutyrate enzymatically (EC 4.3.1.19). For example, the enzyme can be threonine ammonia-lyase from E. coli or Corynebacterium glutamicum. The 2-oxobutyrate is converted to propionyl-CoA enzymatically (EC 1.2.7.1). For example, the enzyme can be pyruvate synthase from Methanosarcina barkeri (UNIPROT P80521, P80522, P80523, and P80524) or Aeropyrum pernix (UNIPROT Q9YA13 and Q9YA11). In another embodiment, the host organism overproduces homoserine or methionine. In this example homoserine or methionine is converted to 2-oxobutyrate enzymatically (EC 4.4.1.11). The enzyme can be methionine gamma-lyase from Pseudomonas putida. The 2-oxobutyrate is converted to propionyl-CoA enzymatically (EC 1.2.7.1). The enzyme can be pyruvate synthase from Methanosarcina barkeri (UNIPROT P80521, P80522, P80523, and P80524) or Aeropyrum pernix (UNIPROT Q9YA13 and Q9YA11). The propionyl-CoA resulting from any above embodiment is converted to methylmalonic acid as described in Example 4.

The microorganism expresses all enzymes necessary to convert the amino acid, such as threonine, homoserine or methionine, to methylmalonic acid. The DNA fragments encoding CoA carboxylase (FIG. 5, step A) and CoA hydrolase (FIG. 5, step B) is cloned into an expression vector. The resulting plasmid that successfully transcribes all pathway genes is transformed into a recombinant microorganism that produces the amino acid as described above.

### Example 6—Construction of Recombinant Microorganism for Production of Methylenemalonic Acid

The microorganism used for production of methylenemalonic acid can be selected from fungi, including yeast and filamentous fungi as well as bacteria. The microorganism expresses all enzymes necessary to convert serine to methylenemalonic acid. The DNA fragments encoding a transaminase, a synthase, an alcohol dehydrogenase, a semialdehyde dehydrogenase, a dehydratase, and decarboxylase (FIG. 6) is cloned into an expression vector. The resulting plasmid that successfully transcribes all pathway genes is transformed into a recombinant microorganism that produces serine. The microorganism described in Pharkya et al. can be used as a starting serine overproducing strain for subsequence genetic engineering steps in instances were bacterial production is desired. Similarly, the microorganism described in Stolz et al. and U.S. Ser. No. 00/603,7154A can be used as a starting strain for subsequent genetic engineering steps in instances where eukaryotic production is desired.
Pharkya, Burgard, and Maranas. Exploring the overproduction of amino acids using the bilevel optimization framework optknock. Wiley Intersciences. 24 Nov. 2003.
Stolz et al. Reduced folate supply as key to enhanced L-serine production by Corynebacterium glutamicum. Appliced and Environ. Microbio. February 2007, p. 750-755.

To construct a hydroxypyruvate overproducing microorganism, the serC (Uniprot P23721) gene which codes for phosphoserine aminotransferase is deleted. The serC deletion will result in overproduction of 3-phospho hydroxypyruvate, which will be converted by yeaB or GPP2 to hydroxypyruvate. This genetic strategy is used to construct a starting strain for subsequent genetic engineering steps in instances where either bacterial or eukaryotic production is desired. The hydroxypyruvate overproducing organism described here may be used as an alternative to the serine overproducing organism described above. The microorganism expresses all enzymes necessary to convert hydroxypyruvate to methylenemalonic acid. The DNA fragments encoding a synthase, an alcohol dehydrogenase, a semialdehyde dehydrogenase, a dehydratase, and decarboxylase (FIG. 6) is cloned into an expression vector. The resulting plasmid that successfully transcribes all pathway genes is transformed into a recombinant microorganism that produces hydroxypyruvate.
Ho, Noji, and Saito. Plastidic pathway of serine biosynthesis. Molecular cloning and expression of 3-phosphoserine phosphatase from Arabidopsis thaliana. J Biol chem. 1999 Apr. 16; 274(16):11007-12.

Strains that overproduce itatartaric and culture conditions are described in Jakubowska et al, 1974; Guevarra and Tabuchi, 1990 a and b; and Geiser et al, 2014. In another iteration, the DNA fragment encoding an itaconic oxidase is overproduced. The itaconic oxidase gene is from Aspergillus or Ustilago (Jakubowska et al, 1974; Guevarra and Tabuchi, 1990 a and b; Geiser et al, 2014). The resulting plasmid successfully transcribes all pathway genes for production of alpha (hydroxymethyl) malic acid, also referred to as itatartaric acid. Mutant forms of the itaconic oxidase gene display increased activity (Aprai, 1958; Aprai, 1959; Jakubowska et al., 1967). The lactone form, hydroxyparaconic acid, is also produced. In one example, plasmid expressing genes necessary for itaconic conversion to itatartaric is transformed into an itaconic overproducting host. For example, Aspergillus and Ustilago strains are used as the host, such as Aspergillus terreus, Aspergillus niger, Ustilago cynodontis, or Ustilago maydis. The itaconic oxidase activity occurs naturally from the wild type enzyme, from overexpression of the wild type gene, or from expression of mutant itaconic oxidase gene. An engineered E. coli that overproduces itaconic acid, as described in Vuoristo et al 2014, could be transformed with the itaconic oxidase gene to produce itatartaric acid.

The microorganism expresses all enzymes necessary to convert itatartaric acid to methylenemalonic acid. The DNA fragments encoding an alcohol dehydrogenase, a semialdehyde dehydrogenase, a dehydratase, and decarboxylase (FIG. 6) is cloned into an expression vector. The resulting plasmid that successfully transcribes all pathway genes is transformed into a recombinant microorganism that produces itatartaric acid.

Itaconic acid oxidase activity can be detected using any method known in the art. For example, the assay described in Geiser et al can be used to determine itaconic oxidase activity by detected the product itatartaric acid via HPLC assay.

Ustilago maydis and Aspergillus terreus were grown in defined media for up to 9 days at 30° C. The growth media consisted of 120 g glucose, 1 g urea, 0.2 g KH₂PO₄, 1 g MgSO₄*7H₂O, 1 g yeast extract, 1 mL of 1000× trace metal solution per 1 liter adjusted to the indicated pH. The 1000× trace metal solution was made by addition of 0.125 g ZnSO₄ and 1.25 g FeSO₄*7H₂O to 250 mL water. U. maydis was grown in pH 3, pH 5, and pH 7 medias, while A. terreus was grown in pH 3 media. Time points were taken approximately every 24 hours, and the supernatant was analyzed via HPLC. Itatartaric acid was observed to be predominantly present in its lactone form, hydroxyparaconic acid (HP). Levels of HP product were estimated by comparison with different amounts of synthesized ITT/HP standard. Both Ustilgo maydis and Aspergillus terreus produced HP.
Aprai. Itaconic oxidase: an enzyme from an ultraviolet-induced mutant of Aspergillus terreus. Nature, 1958, 182, 661-662.
Arpai. Ultraviolet-induced mutational changes in enzyme activity of Aspergillus terreus. Journal of Bacteriology, 1959, 78, 153-158.
Geiser, Wiebach, Wierckx, and Blank. Prospecting the biodiversity of the fungal family Ustilaginaceae for the production of value-added chemicals. Fulgal Biology and Biotechnology 2014, 1:2.
Guevarra and Tabuchi. Accumulation of Itaconic, 2-hydroxyparaconic, itatartaric, and malic acids by strains of the genus Ustilago. Agric. Biol. Chem. 1990, 54 (9), 2353-2358.
Guevarra and Tabuchi. Production of 2-hydroxyparaconic and itatartaric acids by Ustilago cynodontis and simple recovery process of the acids. Agric. Biol. Chem., 1990, 54 (9), 2359-2365.
Jakubowska and Metodiewa. Studies on the metabolic pathway for itatartaric acid formation by Aspergillus terreus II. Use of (−)-citramalate, citraconate and itaconate by cell-free extracts. Acta Microbiologica Polonica Ser. B 1974, Vol. 6 (23), No. 2, 51-61.
Jakubowska, Oberman, Makiedonska, and Florianowicz. The itatonic and itatartaric acid formation by uv-and gamma-irradiated isolates of Aspergillus terreus NRRL 1960. 1967, 16(1), 53-68.
Vuuoristo et al. Metabolic engineering of itaconate production in Escherichia coli. Appl Microbiol Biotechnol, July 2014.

Examples of enzymes (and their corresponding references) to convert serine, hydroxypyruvate, and/or itatartaric acid to methylenemalonic acid are shown in Table 2 and are described in the accompanying text below. The resulting plasmid that successfully transcribes all pathway genes is transformed into a recombinant microorganism that produces serine, hydroxypyruvate and/or itatartaric acid as described above. Assays to demonstrate enzymatic activity of are described below.

Additionally, expression of a DNA fragment encoding a methylenemalonic acid transporter improves production of methylenemalonic acid. For example, the transporter gene may be msfA encoding the putative Major Facilitator Superfamily protein from Aspergillus terreus (UNIPROT Q0C8L2), or homologs thereof.

TABLE 2
Enzyme for production of methylenemalonic acid.

| Enzyme category | EC Number | Enzyme name | Organism |
|---|---|---|---|
| transaminase | 2.6.1.1 | aspartate transaminase | E. coli |
| | 2.6.1.42 | branched-chain-amino-acid | Schizosaccharomyces |
| | | transaminase | pombe |
| | 2.6.1.45 | serine-glyoxylate transaminase | Arabidopsis thaliana |
| synthase | 2.3.3.13 | 2-isopropylmalate synthase | Arabidopsis thaliana |
| | 2.3.3.14 | homocitrate synthase | Saccharomyces |
| | | | cerevisiae |
| | 2.3.3.14 | homocitrate synthase | Schizosaccharomyces |
| | | | pombe |
| | 2.3.3.14 | homocitrate synthase | Azobacter vinelandii |
| | 2.3.3.14 | homocitrate synthase | Lotus japonicus |
| alcohol | 1.1.1.1 | alcohol dehydrogenase | Saccharomyces |
| dehydrogenase | | | cerevisiae |
| | 1.1.1.31 | 3-hydroxyisobutyrate | Bacillus cereus |
| | | dehydrogenase | |
| | 1.1.1.31 | 3-hydroxyisobutyrate | Homo sapiens |
| | | dehydrogenase | |
| semialdehyde | 1.2.1.79 | succinate-semialdehyde | E. coli |
| dehydrogenase | | dehydrogenase | |
| | 1.2.1.79 | succinate-semialdehyde | Sulfolobus solfataricus |
| | | dehydrogenase | |
| | 1.2.1.3 | aldehyde dehydrogenase | Saccharomyces |
| | | | cerevisiae |
| dehydratase | 4.2.1.3 | aconitate hydratase | E. coli |
| | 4.2.1.3 | aconitate hydratase | Saccharomyces |
| | | | cerevisiae |
| | 4.2.1.31 | maleate hydratase | Methanocaldococcus |
| | | | jannaschii |
| | 4.2.1.33 | 3-isopropylmalate dehydratase | Saccharomyces |
| | | | cerevisiae |
| decarboxylase | 4.1.1.6 | cis-aconitate decarboxylase | Aspergillus terreus |
| | 4.1.1.6 | cis-aconitate decarboxylase | Aspergillus niger |
| | 4.1.1.6 | cis-aconitate decarboxylase | Mus musculus |

Transaminase Activity Assay

A person skilled in the art will appreciate that the activity of many transaminase enzymes has been characterized and that any method known in the art for detecting transaminase activity can be used. Upon expression of the Arabidopsis thaliana transaminase that activity can be characterized using the assay described by Kendziorek and Paszkowski. The amount of reaction using glycine as the amino group donor is estimated by determining the remaining 2-oxoacid substrate after the reaction was stopped, which is determined by a spectrophotometric method using NADH and lactate dehydrogenase.
Kendziorek and Paszkowski. Properties of serine:glyoxylate aminotransferase purified from Arabidopsis thaliana leaves. Acta Biochim Biophys Sin, 2008, 40 (2): 102-110.

Synthase Activity Assay

E. coli optimized genes encoding synthases were synthesized and cloned into pTrcHisA (Life Technologies (formerly Invitrogen)). Synthase genes tested are found in Tables 3 and 4. Plasmids containing the optimized synthase genes were transformed into BL21 E. coli cells. Empty plasmid pTrcHisA was also transformed as a negative control. For expression and characterization experiments, shake flasks containing 40 mL TB were inoculated at 5% from overnight cultures. Flasks were incubated at 30° C. at 250 rpm shaking for 2 hours, then protein production was induced with 0.2 mM IPTG and incubated for 4 more hours at 30° C. while shaking. Cells were harvested by centrifugation and pellets were stored at −80° C.

Activity of synthase candidates was assessed with an in vitro assay using DTNB (5,5′-dithiobis(2-nitrobenzoic acid)) as an indicator. The enzyme activity was tested using either no substrate or hydroxypyruvate as the substrate. The DTNB interacts with the free thio created by the condensation of acetyl-CoA and the substrate present. Unless otherwise specified, all chemicals were purchased from Sigma-Aldrich Chemical Company, St. Louis, Mo.

Cells were lysed using mechanical disruption using a BeadBeater (BopSpec products, Bartlesville, Okla.) following the manufacturer's instructions. The cell lysate was partially clarified by centrifugation (14,000 G for 5 minutes). Protein concentrations of the resulting clarified lysates were measured via BioRad total Protein assay using the manufacturer's instructions. Lysates were normalized by protein concentration in 100 mM Tris buffer. The normalized lysates were diluted 1 to 7 in 100 mM Tris buffer. A 20 μL volume of lysate was added to each well for the 96-well plate assay. Each condition was performed in triplicate.

The reaction mixture contains 100 mM Tris pH 7.4, 5 mM MgSO₄, 0.2 mM acetyl-CoA, 0.5 mM DTNB, 0.5 mM substrate, hydroxypyruvate. To start the reaction, 180 μL of reaction mix was added to each well already containing 20 μL lysate. The reactions in these microplates were monitored at 412 nm. Readings were taken every 9 seconds for 10 minutes and the data was used to calculate activities of each enzyme. Synthase activity was observed when hydroxypyruvate was the substrate as compared to cells containing empty vector (Table 3). Background absorbance as measured by the same reaction with no substrate present were subtracted. Error bars in the graphs reflect the standard deviations calculated for the averages for each condition performed in triplicate. Specific mutations change the activity of the enzymes tested (Table 4). Further enzyme engineering will improve specificity and activity of desired enzymatic reaction.

TABLE 3
List of candidate synthases and activity with hydroxypyruvate.

| | | | GenBank | Activity with | |
|---|---|---|---|---|---|
| | | | Accession | hydroxy pyruvate | |
| Name | Gene | Organism | Number | (μmol/min/mg) | stdev |
| EV | empty | | | 0.016 | 0.009 |
| | vector, ptrc | | | | |
| ScLys20 | lys20 | Saccharomyces | CAA58264 | 0.095 | 0.057 |
| | | cerevisiae | | | |
| PcLys1 | lys1 | Penicillium | CAP98607 | 0.008 | 0.001 |
| | | chrysogenum | | | |
| SpLys4 | Lys4 | Schizosaccharomyces | CAB50965 | 0.034 | 0.016 |
| | | pombe | | | |
| TtHCS | HCS | Thermus thermophilis | AAS81892 | 0.025 | 0.009 |
| AvNifV | NifV | Azotobacter vinelandii | AAA22169 | 0.054 | 0.011 |
| AtMamL | mamL | Arabidopsis thaliana | CAC80102 | 0.015 | 0.005 |
| | (mam1) | | | | |
| AtMam3 | mam3 | Arabidopsis thaliana | AED93108 | 0.009 | 0.001 |
| MtAksA | AksA | Methanothermobacter | AAB86103 | 0.011 | 0.004 |
| | | thermautotrophicus | | | |
| LiLeuA | LeuA/CimA | Leptospira interogans | AAN49401 | 0.045 | 0.005 |
| SeLeuA | LeuA | Salmonella enterica | X51583 | 0.004 | 0.001 |
| EcLeuA | LeuA | Escherichia coli | AAC73185 | 0.022 | 0.005 |
| LjFen1 | FEN1 | Lotus japonicus | BAI49592 | 0.038 | 0.001 |
| AtIPMS1 | IPMS1 | Arabidopsis thaliana | AEE29723 | 0.041 | 0.002 |
| SpIPMS1 | IPMS1 | Schizosaccharomyces | CAW33849 | 0.016 | 0.002 |
| | | pombe | | | |

TABLE 4
List of mutant candidate synthases and activity with hydroxy
pyruvate and comparison with wild type synthase

| | | | | Activity | |
|---|---|---|---|---|---|
| | | | | with hydroxy | |
| Name | Gene | Organism | Mutation | pyruvate | stdev |
| EV | empty vector, | | | 0.016 | 0.009 |
| | ptrc | | | | |
| TtHCS | HCS | Thermus thermophilis | | 0.025 | 0.009 |
| TtHCSmt1 | HCS mutant - | Thermus thermophilis | H72L | 0.035 | 0.002 |
| | H72L | | | | |
| SpLys4 | Lys4 | Schizosaccharomyces | | 0.034 | 0.016 |
| | | pombe | | | |
| SpLys4mt1 | Lys4 mutant - | Schizosaccharomyces | D123N | 0.004 | 0.003 |
| | D123N | pombe | | | |
| SpLys4mt2 | Lys4 mutant - | Schizosaccharomyces | D123N, V125F | 0.009 | 0.003 |
| | D123N, V125F | pombe | | | |
| SpLys4mt3 | Lys4 mutant - | Schizosaccharomyces | D123N, | 0.013 | 0.004 |
| | D123N, V125F, | pombe | V125F, I194L | | |
| | I194L | | | | |

Dehydrogenase Assay

E. coli optimized genes encoding dehydrogenases are synthesized and cloned into pTrcHisA (Life Technologies (formerly Invitrogen)). Dehydrogenase candidates are found in Table 2. Plasmids containing the optimized dehydrogenase genes are transformed into BL21 E. coli cells. Empty plasmid pTrcHisA are also transformed as a negative control. For expression and characterization experiments, shake flasks containing 40 mL TB are inoculated at 5% from overnight cultures. Flasks are incubated at 30° C. at 250 rpm shaking for 2 hours, then protein production is induced with 0.2 mM IPTG and incubated for 4 more hours at 30° C. while shaking. Cells are harvested by centrifugation and pellets are stored at −80° C.

Activity of dehydrogenase candidates is assessed with an in vitro assay using the conversion of the co-factor NAD⁺ to NADH as measured at 340 nm with a UV-vis spectrophotometer. The enzyme activity is tested using either no substrate or in the presence of substrate. In the case of the alcohol dehydrogenase reaction, the substrate is itatartaric acid. In the semialdehyde dehydrogenase reaction, the substrate is 1-formyl-1-hydroxy-1,2-ethanedicarboxylic acid. The formation of NADH causes an increase in absorption at 340 nm. Unless otherwise specified, all chemicals are purchased from Sigma-Aldrich Chemical Company, St. Louis, Mo.

Cells are lysed using mechanical disruption using a BeadBeater (BopSpec products, Bartlesville, Okla.) using the manufacturer's instructions. The cell lysate is partially clarified by centrifugation (14,000 G for 5 minutes). Protein concentrations of the resulting clarified lysates are measured via Pierce 660 nm total Protein assay using the manufacturer's instructions. Lysates are normalized by protein concentration in 100 mM potassium phosphate buffer. The normalized lysates are diluted 1 to 10 in 100 mM phosphate buffer. 10 μL of lysate was added to each well for the 96-well plate assay. Each condition was performed in triplicate.

The reaction mixture contains 100 mM potassium phosphate buffer pH 6.8, 20 mM substrate. To start the reaction, 70 μL of reaction mix is added to each well already containing 30 μL lysate. The reactions in these microplates are monitored at 340 nm. Readings are taken every 10 seconds for 20 minutes and the data is used to calculate activities of each enzyme.

Background absorbance as measured by the same reaction with no substrate present are subtracted.

The same reactions are allowed to incubate overnight at 30° C. The samples are boiled for 5 min at 100° C. to denature the protein. The samples are centrifuged to remove the protein debris and the resulting supernatant is analyzed by HPLC to measure formation of the desired product.

Dehydratase Assay

E. coli optimized genes encoding dehydratases are synthesized and cloned into pTrcHisA (Life Technologies (formerly Invitrogen)). Dehydratase candidates are found in Table 5. Plasmids containing the optimized dehydratase genes are transformed into BL21 E. coli cells. Empty plasmid pTrcHisA are also transformed as a negative control. For expression and characterization experiments, shake flasks containing 40 mL TB are inoculated at 5% from overnight cultures. Flasks are incubated at 30° C. at 250 rpm shaking for 2 hours, then protein production is induced with 0.2 mM IPTG and incubated for 4 more hours at 30° C. while shaking. Cells are harvested by centrifugation and pellets are stored at −80° C.

TABLE 5
Dehydratase candidates.

| | | GenBank |
|---|---|---|
| Gene | Organism | number |
| AcoA | Aspergillus nidulans | AAN61439 |
| Aco1 | Yarrowia lipolytica | AAT92542 |
| Aco1 | Saccharomyces cerevisiae | AAA34389 |
| Aco2 | Saccharomyces cerevisiae | CAA54757 |
| Leu2 | Saccharomyces cerevisiae | CAA27459 |
| TthacAB | Thermus thermophilus | BAA74762, |
| | | BAA74763 |
| Aco | Sulfolobus acidocaldarius | AEG71149 |
| Aco | Sus scrofa | AAA30987 |
| AcnA | E. coli | CAA42834 |
| AcnB | E. coli | AAC73229 |
| AcoA | Aspergillus fumigatus | EAL89133 |

Activity of dehydratase candidates is assessed with an in vitro assay using the conversion of a single bond in the substrate to a double bond in the product measured at 235 nm with a UV-vis spectrophotometer. The enzyme activity is tested using either no substrate or 1-hydroxy-1,1,2-ethanetricarboxylic acid, as the substrate. The formation of the double bond causes an increase in absorption at 235 nm. The reaction can also be tested in the opposite direction, double bond to single bond, which results in a decrease in absorption at 235 nm. Either forward or reverse will give information to be able to calculate activity of the dehydratase candidate for the desired reaction. Unless otherwise specified, all chemicals are purchased from Sigma-Aldrich Chemical Company, St. Louis, Mo.

Cells are lysed using mechanical disruption using a BeadBeater (BopSpec products, Bartlesville, Okla.) using the manufacturer's instructions. The cell lysate is partially clarified by centrifugation (14,000 G for 5 minutes). Protein concentrations of the resulting clarified lysates are measured via BioRad total Protein assay using the manufacturer's instructions. Lysates are normalized by protein concentration in 100 mM TAPS buffer. The normalized lysates are diluted 1 to 10 in 100 mM TAPS buffer. 10 μL of lysate was added to each well for the 96-well plate assay. Each condition was performed in triplicate.

The reaction mixture contains 100 mM TAPS buffer pH 6.8, 100 mM KCl, 100 mM substrate alpha-hydroxymethyl maleic acid. The dehydratase lysates are incubated in the presence of 1 mM ammonium ferrous sulphate and 5 mM DTT to reconstitute the iron-sulfur cluster of the enzyme for 30 minutes. To start the reaction, 90 μL of reaction mix is added to each well already containing 10 μL lysate. The reactions in these microplates are monitored at 235 nm. Readings are taken every 9 seconds for 10 minutes and the data is used to calculate activities of each enzyme. Background absorbance is measured by the same reaction with no substrate present are subtracted.

The same reactions are allowed to incubate overnight at 30° C. The samples are boiled for 5 min at 100° C. to denature the protein. The samples are centrifuged to remove the protein debris and the resulting supernatant is analyzed by HPLC to measure formation of the desired product.

Decarboxylase Activity Assay

E. coli optimized genes encoding decarboxylases are synthesized and cloned into pTrcHisA (Life Technologies (formerly Invitrogen)). Decarboxylase candidates are found in Table 6. Plasmids containing the optimized synthase genes were transformed into BL21 E. coli cells. Empty plasmid pTrcHisA is also transformed as a negative control. For expression and characterization experiments, shake flasks containing 40 mL TB are inoculated at 5% from overnight cultures. Flasks are incubated at 30° C. at 250 rpm shaking for 2 hours, then protein production is induced with 0.2 mM IPTG and incubated for 4 more hours or overnight at 30° C. while shaking. Cells are harvested by centrifugation and pellets were stored at −80° C.

Activity of decarboxylase candidates are assessed with an in vitro lysate assay whereas the acrylate product is detected using HPLC. The enzyme activity is tested using either no substrate or the alpha substituted maleic as the substrate. The acrylate product is detected using Benson organic acid column (300×7.8 mm, Part #2000-0 BP-OA) and run using 2 Benson columns in tandem, 4% acetonitrile+0.025 N sulfuric acid mobile phase. Unless otherwise specified, all chemicals are purchased from Sigma-Aldrich Chemical Company, St. Louis, Mo.

Cells are lysed using mechanical disruption using a BeadBeater™ (BopSpec products, Bartlesville, Okla.) using the manufacturer's instructions. The cell lysate is partially clarified by centrifugation (14,000 G for 5 minutes). Protein concentrations of the resulting clarified lysates are measured via BioRad total Protein assay using the manufacturer's instructions. Lysates are normalized by protein concentration in 100 mM sodium phosphate buffer, pH 6.3.

The reaction mixture contains 100 mM Sodium phosphate buffer pH 6.3, 1 μL DTT, and 10 mM substrate alpha (substituted) maleic acid. The reactions are allowed to incubate overnight at 30° C. The samples are boiled for 5 min at 100° C. to denature the protein. The samples are centrifuged to remove the protein debris and the resulting supernatant is analyzed by HPLC to measure formation of the desired product. Decarboxylase activity is observed with substrate as compared to cells containing empty vector.

TABLE 6
List of Exemplary decarboxylase sequences

| | | GenBank/ |
|---|---|---|
| | | Accession |
| Gene | Organism | number |
| CadA (cis-aconitate | Aspergillus terreus | BAG49047 |
| decarboxylase A) | | |
| CadA (cis-aconitate | Aspergillus niger | EAU29420 |
| decarboxylase A) | | |
| Stipitatonate | Talaromyces stipitatus | XP_002341280 |
| Decarboxylase | | |
| FDC1 (Ferulic acid | Saccharomyces cerevisiae | AAB64981 |
| decarboxylase 1) | | |
| MmgE/PrpD family | Halarchaeum acidiphilum | WP_021779749 |
| protein | | |
| MmgE/PrpD family | Cupriavidus sp. HMR-1 | WP_008644277 |
| protein | | |
| CadA (cis-aconitate | Mus Musculus | BAC29433 |
| decarboxylase A) | | |
| 4-oxalocrotonate | Geobacillus | ACA01540 |
| decarboxylase | stearothermophilus | |
| 4-oxalocrotonate | Pseudomonas putida | AAA25693 |
| decarboxylase | | |
| 2-hydroxymuconate-6- | Pseudomonas putida | AAA26054 |
| semialdehyde hydrolase | | |
| Phosphoenolpyruvate | Saccharomyces cerevisiae | CAA31488 |
| carboxykinase (ATP) | | |
| MmgE/prpD family | Aspergillus terreus | XP_001215146 |
| protein | NIH2624 | |
| MmgE/prpD family | Bacillus subtilis | BAA08333 |
| protein | | |
| MmgE/prpD family | Lactobacillus sucicola | GAJ27510 |
| protein | JCM 15457 | |
| MmgE/prpD family | Bordetella pertussis | NP_881740 |
| protein | Tohama I | |
| MmgE/prpD family | Bordetella pertussis | NP_878944 |
| protein | Tohama I | |
| MmgE/prpD family | Bacillus firmus DS1 | EWG10287 |
| protein | | |
| MmgE/prpD family | Rhodococcus opacus | AHK34564 |
| protein | PD630 | |
| MmgE/prpD family | Rhodococcus rhodochrous | WP_016693543 |
| protein | | |

### Example 7—Fermentation

Fed-batch fermentation is performed in a 2 L working capacity fermenter. Temperature, pH and dissolved oxygen are controlled by PID control loops. Temperature is maintained at 37° C. by temperature adjusted water flow through a jacket surrounding the fermenter vessel at the growth phase, and later adjusted to 27° C. when production phase started. The pH is maintained at the desired level by the addition of 5 N KOH and 3 N H₃PO₄. Dissolved oxygen (DO) level is maintained at 20% of air saturation by adjusting air feed as well as agitation speed.

Inoculant is started by introducing a single colony picked from an LB agar plate into 50 mL TB medium. The culture is grown at 37° C. with agitation at 250 rpm until the medium is turbid. Subsequently a 100 mL seed culture is transferred to fresh M9 glucose medium. After culturing at 37° C. and 250 rpm for an additional 10 h, an aliquot (50 mL) of the inoculant (OD600=6-8) is transferred into the fermentation vessel and the batch fermentation was initiated. The initial glucose concentration in the fermentation medium is about 40 g/L.

Cultivation under fermentor-controlled conditions is divided into two stages. In the first stage, the airflow is kept at 300 ccm and the impeller speed is increased from 100 to 1000 rpm to maintain the DO at 20%. Once the impeller speed reaches its preset maximum at 1000 rpm, the mass flow controller starts to maintain the DO by oxygen supplementation from 0 to 100% of pure O₂.

The initial batch of glucose is depleted in about 12 hours and glucose feed (650 g/L) is started to maintain glucose concentration in the vessel at 5-20 g/L. At OD600=20-25, IPTG stock solution is added to the culture medium to a final concentration of 0.2 mM. The temperature setting is decreased from 37 to 27° C. and the production stage (i.e., second stage) is initiated. Production stage fermentation is run for 48 hours and samples are removed to determine the cell density and quantify metabolites. Production of specific products is measured by GS/MS.

### Example 8—Separation of Fermentation Products, Including 2-Hydroxymethyl Malonic Acid, Methyltartronic Acid, and Methylmalonic Acid

Fermentation broth containing dicarboxylic acid fermentation products, including hydroxymethyl malonic acid, methyltartronic acid, and methylmalonic acid, are separated using methods developed for various carboxylic acids. Such methods include separation using anion exchange, ultra-filtration, distillation, electro-dialysis, reverse osmosis, and various extraction methods as reviewed in Kumar and Babu 2008.
Kumar and Babu. Process intensification for separation of carboxylic acids from fermentation broths using reactive extraction. Journal on Future Engineering & Technology, Vol. 3(3), pp 19.26.

### Example 9—Method of Converting 2-Hydroxymethylmalonic Acid to Methylenemalonic Acid

The conversion of 2-hydroxymethylmalonic acid to methylenemalonic acid is performed via methods similar to those used to convert 3-hydroxypropionic acid to acrylic acid. Examples are found in U.S. Pat. Nos. 7,538,247, 9,029,596, 8,338,145 and 9,181,170. In one instance, the conditions of conversion are 60% 3-HP in water, 250° C. (vapor phase) in the presence of γ-alumina catalyst. In another embodiment, the conditions of conversion are 300 C (vapor phase), 12% 3-HP in water in the presence of silica alumina (JGC Corp.) catalyst. The conditions of conversion may also include a 4:1 ratio of 3-HP to H₂SO₄, 30% 3-HP in water in the presence of sulfuric acid, catalysis in a GC column.

An example of conversion of a structurally similar analog is dehydration of alpha-substituted 3-hydroxypropionic acid. This compound is dehydrated to alpha-substituted acrylic acid. In one embodiment, alpha-hydroxymethyl-3-hydroxypropionic acid (HM3HP) is dehydrated to alpha-hydroxymethyl acrylic acid (HMA). A known amount of HM3HP was dissolved into a buffered solution. The solution was split into three aliquots which were adjusted to pH 3, pH 7, or pH 10. Samples were incubated at −20° C., 30° C., or 70° C. overnight. NMR analysis was used to measure the amount of HM3HP that was dehydrated to HMA. The most conversion to HMA was observed at the pH 10 (Table 7). The results indicate that a more basic pH drives conversion of HM3HP to HMA. The pH of the solution had more effect on conversion to HMA than did changes in temperature.

TABLE 7
HMA converted from HM3HP at different temperatures and pH

| | | Relative |
|---|---|---|
| | Temperature | HMA levels |
| pH 3 | −20° C. | 0.00 |
| | 30° C. | 0.00 |
| | 70° C. | 0.00 |
| pH 7 | −20° C. | 0.13 |
| | 30° C. | 0.13 |
| | 70° C. | 0.18 |
| pH 10 | −20° C. | 0.81 |
| | 30° C. | 1.18 |
| | 70° C. | 1.00 |

### Example 10—Method for Converting Methyltartronic Acid to Methylenemalonic Acid

The conversion of methyltartronic acid to methylenemalonic acid is performed via methods similar to those used to convert lactic acid to acrylic acid. Examples are found in US2012078004A1 and U.S. Pat. No. 9,260,550B1. In one embodiment, the conversion conditions may be 250° C., 300 psig in the presence of a homogenous nickel catalyst, followed by pyrolysis. In another example, the conversion conditions may be reaction with bromination material (for example N-bromosuccinimide), followed by further reaction with an elimination material (for example trimethylamine).

### Example 11—Method for Converting Methylmalonic Acid to Methylenemalonic Acid

The conversion of methylmalonic acid to methylenemalonic acid is performed via methods similar to those used to convert isobutyric acid to methacrylic acid. Examples are found in U.S. Pat. Nos. 5,618,974, 5,335,954, and Bonnet et al, 1996. For instance, the conversion conditions may be 270° C., 5% isobutyric, 10% O₂, 10% steam, 75% N₂, 2000 h⁻¹ space velocity in the presence of a powder catalyst with Vn, Mo, P, and As. Relevant methods of dehydrogenation including oxidative dehydrogenation and catalytic dehydrogenation are reviewed in Weissermel and Arpe, 2008.
Bonnet, P., et al. “Study of a new iron phosphate catalyst for oxidative dehydrogenation of isobutyric acid.” Journal of Catalysis 158.1 (1996): 128-141.
Weissermel and Arpe. Industrial Organic Chemistry. John Wiley & Sons, Jul. 1, 2008, Science, 481 pages.

### Example 12—Method of Fermenting and Separating Methylenemalonic Acid

Fermentation methods for the production of methylenemalonic acid or an intermediate thereof are carried out as described in Example 7. Their separation is performed via methods similar to those used to separate itaconic acid from fermentation broth, for instance, anion exchange, reverse osmosis, crystallization, membrane extraction, and/or vaporization (U.S. Pat. No. 3,544,455A, CN 102940992A, CN 101643404B). For example, methods to separate prepared methylenemalonic acid are described in reference WO2012054633A2, U.S. Pat. Nos. 3,758,550, and 2,313,501.

1. A recombinant microorganism comprising 2-hydroxymethylmalonic acid, and at least one recombinant nucleic acid sequence encoding at least one enzyme selected from a CoA carboxylase and a CoA hydrolase, wherein the 2-hydroxymethylmalonic acid is a compound of Formula II: 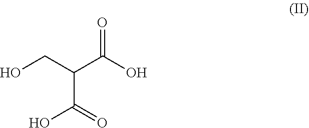
or a salt or ester thereof.

2. The recombinant microorganism according to claim 1, wherein the recombinant microorganism further comprises 3-hydroxypropionyl-CoA or a salt or ester thereof.

3. The recombinant microorganism according to claim 1 or 2, wherein the recombinant microorganism selectively overproduces 2-hydroxymethylmalonic acid, or a salt or ester thereof.

4. The recombinant microorganism according to any one of claims 1 to 3, wherein the recombinant microorganism produces at least 0.1 g/L/hour of 2-hydroxymethylmalonic acid or a salt or ester thereof.

5. The recombinant microorganism according to claim 4, wherein the recombinant microorganism produces at least 0.1 g/L/hour of 2-hydroxymethylmalonic acid.

6. The recombinant microorganism according to any one of claims 1 to 5, further comprising a recombinant nucleic acid sequence encoding an organic acid transporter.

7. The recombinant microorganism according to any one of claims 1 to 6, wherein the recombinant microorganism is a prokaryote.

8. The recombinant microorganism according to any one of claims 1 to 6, wherein the microorganism is selected from Escherichia coli (E. coli), Enterobacter, Azotobacter, Erwinia, Bacillus, Pseudomonas, Klebsiella, Proteus, Salmonella, Serratia, Shigella, Rhizobia, Vitreoscilla, and Paracoccus.

9. The recombinant microorganism according to any one of claims 1 to 6, wherein the recombinant microorganism is a eukaryote.

10. The recombinant microorganism of claim 9, wherein the recombinant microorganism is a yeast or a fungus.

11. The recombinant microorganism according to any one of claims 1 to 6, wherein the microorganism is selected from Candida, Pichia, Saccharomyces, Schizosaccharomyces, Zygosaccharomyces, Kluyveromyces, Debaryomyces, Pichia, Issatchenkia, Yarrowia, Hansenula, Aspergillus and Ustilago.

12. The recombinant microorganism according to claim 11, wherein said microorganism is a host yeast cell selected from C. sonorensis, K. marxianus, K. thermotolerans, C. methanesorbosa, Saccharomyces bulderi (S. bulderi), I. orientalis, C. lambica, C. sorboxylosa, C. zemplinina, C. geochares, P. membranifaciens, Z. kombuchaensis, C. sorbosivorans, C. vanderwaltii, C. sorbophila, Z. bisporus, Z. lentus, Saccharomyces bayanus (S. bayanus), D. castellii, C, boidinii, C. etchellsii, K. lactis, P. jadinii, P. anomala, Saccharomyces cerevisiae (S. cerevisiae), Pichia galeiformis, Pichia sp. YB-4149 (NRRL designation), Candida ethanolica, P. deserticola, P. membranifaciens, P. fermentans, Saccharomycopsis crataegensis (S. crataegensis), Aspergillus niger, Aspergillus terreus, Aspergillus oryzae, Ustilago maydis, Ustilago cynodontis, or other fungi.

13. A method for making 2-hydroxymethylmalonic acid or a salt or ester thereof, comprising culturing the recombinant microorganism of any one of claims 1 to 12 in the presence of a carbon source (e.g. a carbohydrate); and isolating the 2-hydroxymethylmalonic acid or its salt or ester.

14. A method for making a methylenemalonic acid of Formula I: 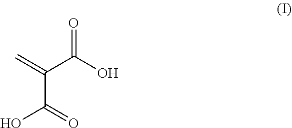
or a salt or ester thereof;
the method comprising treating a compound of Formula II: 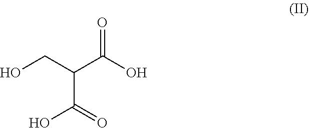
or a salt or ester thereof;
by heating and/or contacting with a catalyst to dehydrate the compound of Formula II to produce a compound of Formula I, or its salt or ester.

15. The method of claim 14, wherein said method further comprises making a compound of Formula II, comprising the steps of culturing the recombinant microorganism of any one of claims 1 to 12 in the presence of a carbon source (e.g. a carbohydrate); and isolating the compound of Formula II.

16. A recombinant microorganism comprising 2,3-dioxobutyric acid or acetoacetic acid or a salt or ester thereof and at least one recombinant nucleic acid sequence encoding at least one enzyme selected from a CoA-hydrolase, a thiolase and an alcohol dehydrogenase.

17. The recombinant microorganism according to claim 16, wherein the recombinant microorganism selectively overproduces 2,3-dioxobutyric acid or acetoacetic acid, or a salt or ester thereof.

18. The recombinant microorganism according to claim 16 or 17, wherein the recombinant microorganism produces at least 0.1 g/L/hour of 2,3-dioxobutyric acid or acetoacetic acid, or a salt or ester thereof.

19. The recombinant microorganism according to claim 18, wherein the recombinant microorganism produces at least 0.1 g/L/hour of 2,3-dioxobutyric acid or acetoacetic acid.

20. The recombinant microorganism according to any one of claims 16 to 19, further comprising a recombinant nucleic acid sequence encoding an organic acid transporter.

21. The recombinant microorganism according to any one of claims 16 to 20, wherein the recombinant microorganism is a prokaryote.

22. The recombinant microorganism according to any one of claims 16 to 20, wherein the microorganism is selected from Escherichia coli (E. coli), Enterobacter, Azotobacter, Erwinia, Bacillus, Pseudomonas, Klebsiella, Proteus, Salmonella, Serratia, Shigella, Rhizobia, Vitreoscilla, and Paracoccus.

23. The recombinant microorganism according to any one of claims 16 to 20, wherein the recombinant microorganism is a eukaryote.

24. The recombinant microorganism of claim 23, wherein the recombinant microorganism is a yeast or a fungus.

25. The recombinant microorganism according to any one of claims 16 to 20, wherein the microorganism is selected from Candida, Pichia, Saccharomyces, Schizosaccharomyces, Zygosaccharomyces, Kluyveromyces, Debaryomyces, Pichia, Issatchenkia, Yarrowia, Hansenula, Aspergillus and Ustilago.

26. The recombinant microorganism according to claim 25, wherein said microorganism is a host yeast cell selected from C. sonorensis, K. marxianus, K. thermotolerans, C. methanesorbosa, Saccharomyces bulderi (S. bulderi), I. orientalis, C. lambica, C. sorboxylosa, C. zemplinina, C. geochares, P. membranifaciens, Z. kombuchaensis, C. sorbosivorans, C. vanderwaltii, C. sorbophila, Z. bisporus, Z. lentus, Saccharomyces bayanus (S. bayanus), D. castellii, C, boidinii, C. etchellsii, K. lactis, P. jadinii, P. anomala, Saccharomyces cerevisiae (S. cerevisiae), Pichia galeiformis, Pichia sp. YB-4149 (NRRL designation), Candida ethanolica, P. deserticola, P. membranifaciens, P. fermentans, Saccharomycopsis crataegensis (S. crataegensis), Aspergillus niger, Aspergillus terreus, Aspergillus oryzae, Ustilago maydis, Ustilago cynodontis, or other fungi.

27. A method for making 2,3-dioxobutyric acid or acetoacetic acid or a salt or ester thereof, comprising culturing the recombinant microorganism of any one of claims 16 to 26 in the presence of a carbon source (e.g. a carbohydrate); and isolating the 2,3-dioxobutyric acid or acetoacetic acid or a salt or ester thereof.

28. A method for making a methylenemalonic acid of Formula I: 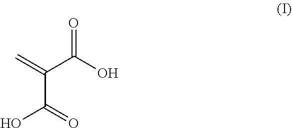
or a salt or ester thereof;
the method comprising treating a methyltartronic acid of Formula III: 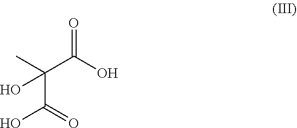
or a salt or ester thereof;
by heating and/or contacting with a catalyst, optionally followed by pyrolysis, to dehydrate the compound of Formula III and/or contacting with a bromination agent followed by an elimination agent such as a base, to produce methylenemalonic acid or a salt or ester thereof.

29. The method of claim 28, wherein said method further comprises preparing methyltartronic acid or a salt or ester thereof, comprising the steps of chemically modifying a 2,3-dioxobutyric acid or acetoacetic acid produced by culturing the recombinant microorganism of any one of claims 16 to 26 in the presence of a carbon source (e.g. a carbohydrate); and isolating the compound of Formula III.

30. A recombinant microorganism comprising methylmalonic acid and at least one recombinant nucleic acid sequence encoding at least one enzyme selected from a CoA carboxylase and a CoA hydrolase, wherein said methylmalonic acid is a compound of Formula IV: 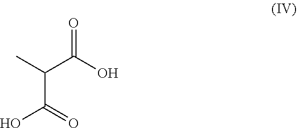
or a salt or ester thereof.

31. The recombinant microorganism according to claim 30, wherein the recombinant microorganism further comprises propionyl-CoA or a salt or ester thereof.

32. The recombinant microorganism according to claim 30, wherein the recombinant microorganism further comprises 2-oxobutyrate or a salt or ester thereof.

33. The recombinant microorganism according to any one of claims 30 to 32, wherein the recombinant microorganism selectively overproduces methylmalonic acid, or a salt or ester thereof.

34. The recombinant microorganism according to any one of claims 30 to 33, wherein the recombinant microorganism produces at least 0.1 g/L/hour of methylmalonic acid, or a salt or ester thereof.

35. The recombinant microorganism according to claim 34, wherein the recombinant microorganism produces at least 0.1 g/L/hour of methylmalonic acid.

36. The recombinant microorganism according to any one of claims 30 to 35, further comprising a recombinant nucleic acid sequence encoding an organic acid transporter.

37. The recombinant microorganism according to any one of claims 30 to 36, wherein the recombinant microorganism is a prokaryote.

38. The recombinant microorganism according to any one of claims 30 to 36, wherein the microorganism is selected from Escherichia coli (E. coli), Enterobacter, Azotobacter, Erwinia, Bacillus, Pseudomonas, Klebsiella, Proteus, Salmonella, Serratia, Shigella, Rhizobia, Vitreoscilla, and Paracoccus.

39. The recombinant microorganism according to any one of claims 30 to 36, wherein the recombinant microorganism is a eukaryote.

40. The recombinant microorganism of claim 39, wherein the recombinant microorganism is a yeast or a fungus.

41. The recombinant microorganism according to any one of claims 30 to 36, wherein the microorganism is selected from Candida, Pichia, Saccharomyces, Schizosaccharomyces, Zygosaccharomyces, Kluyveromyces, Debaryomyces, Pichia, Issatchenkia, Yarrowia, Hansenula, Aspergillus and Ustilago.

42. The recombinant microorganism according to claim 41, wherein said microorganism is a host yeast cell selected from C. sonorensis, K. marxianus, K. thermotolerans, C. methanesorbosa, Saccharomyces bulderi (S. bulden), I. orientalis, C. lambica, C. sorboxylosa, C. zemplinina, C. geochares, P. membranifaciens, Z. kombuchaensis, C. sorbosivorans, C. vanderwaltii, C. sorbophila, Z. bisporus, Z. lentus, Saccharomyces bayanus (S. bayanus), D. castellii, C, boidinii, C. etchellsii, K. lactis, P. jadinii, P. anomala, Saccharomyces cerevisiae (S. cerevisiae), Pichia galeiformis, Pichia sp. YB-4149 (NRRL designation), Candida ethanolica, P. deserticola, P. membranifaciens, P. fermentans, Saccharomycopsis crataegensis (S. crataegensis), Aspergillus niger, Aspergillus terreus, Aspergillus oryzae, Ustilago maydis, Ustilago cynodontis, or other fungi.

43. A method for making methylmalonic acid, or a salt or ester thereof, comprising culturing the recombinant microorganism of any one of claims 28 to 38 in the presence of a carbon source (e.g. a carbohydrate or amino acid); and separating the methylmalonic acid, or it salt or ester.

44. The method of claim 43, wherein said carbon source is an amino acid selected from threonine, homoserine and methionine.

45. A method for making a methylenemalonic acid of Formula I: 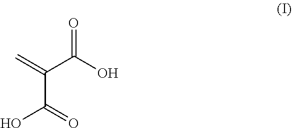
or a salt or ester thereof;
the method comprising treating a compound of Formula IV: 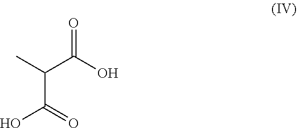
or a salt or ester thereof;
by heating in the presence of O₂ and/or contacting with a catalyst to dehydrogenate the compound of Formula IV to produce the methylenemalonic acid or a salt or ester thereof.

46. The method of claim 45, wherein said method further comprises making a compound of Formula IV, comprising the steps of culturing the recombinant microorganism of any one of claims 30 to 42 in the presence of a carbon source (e.g. a carbohydrate); and separating the compound of Formula IV.

47. A recombinant microorganism comprising methylenemalonic acid or a salt or ester thereof, and at least one recombinant nucleic acid sequence encoding at least one enzyme selected from a transaminase, a synthase, an alcohol dehydrogenase, a semialdehyde dehydrogenase, a dehydratase and a decarboxylase.

48. The recombinant microorganism according to claim 47, wherein the recombinant microorganism further comprises 1,1,2-ethenetricarboxylic acid.

49. The recombinant microorganism according to claim 47 or 48, wherein the recombinant microorganism further comprises 1-hydroxy-1,1,2-ethanetricarboxylic acid.

50. The recombinant microorganism according to any one of claims 47 to 49, wherein the recombinant microorganism selectively overproduces methylenemalonic acid, or a salt or ester thereof.

51. The recombinant microorganism according to any one of claims 47 to 50, wherein the recombinant microorganism produces at least 0.1 g/L/hour of methylenemalonic acid, or a salt or ester thereof.

52. The recombinant microorganism according to claim 51, wherein the recombinant microorganism produces at least 0.1 g/L/hour of methylenemalonic acid.

53. The recombinant microorganism according to any one of claims 47 to 52, further comprising a recombinant nucleic acid sequence encoding an organic acid transporter.

54. The recombinant microorganism according to any one of claims 47 to 53, wherein the recombinant microorganism is a prokaryote.

55. The recombinant microorganism according to any one of claims 47 to 53, wherein the microorganism is selected from Escherichia coli (E. cob), Enterobacter, Azotobacter, Erwinia, Bacillus, Pseudomonas, Klebsiella, Proteus, Salmonella, Serratia, Shigella, Rhizobia, Vitreoscilla, and Paracoccus.

56. The recombinant microorganism according to any one of claims 47 to 53, wherein the recombinant microorganism is a eukaryote.

57. The recombinant microorganism of claim 56, wherein the recombinant microorganism is a yeast or a fungus.

58. The recombinant microorganism according to any one of claims 47 to 53, wherein the microorganism is selected from Candida, Pichia, Saccharomyces, Schizosaccharomyces, Zygosaccharomyces, Kluyveromyces, Debaryomyces, Pichia, Issatchenkia, Yarrowia, Hansenula, Aspergillus and Ustilago.

59. The recombinant microorganism according to claim 58, wherein said microorganism is a host yeast cell selected from C. sonorensis, K. marxianus, K. thermotolerans, C. methanesorbosa, Saccharomyces bulderi (S. bulderi), I. orientalis, C. lambica, C. sorboxylosa, C. zemplinina, C. geochares, P. membranifaciens, Z. kombuchaensis, C. sorbosivorans, C. vanderwaltii, C. sorbophila, Z. bisporus, Z. lentus, Saccharomyces bayanus (S. bayanus), D. castellii, C, boidinii, C. etchellsii, K. lactis, P. jadinii, P. anomala, Saccharomyces cerevisiae (S. cerevisiae), Pichia galeiformis, Pichia sp. YB-4149 (NRRL designation), Candida ethanolica, P. deserticola, P. membranifaciens, P. fermentans, Saccharomycopsis crataegensis (S. crataegensis), Aspergillus niger, Aspergillus terreus, Aspergillus oryzae, Ustilago maydis, Ustilago cynodontis, or other fungi.

60. A method for making methylenemalonic acid or a salt or ester thereof, comprising culturing the recombinant microorganism of any one of claims 47 to 59 in the presence of a carbon source (e.g. a carbohydrate); and isolating the methylenemalonic acid or its salt ester.

61. A compound of the formula: 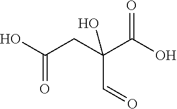 [SMILES: O=CC(O)(CC(=O)O)C(=O)O]
or a salt or ester thereof.
